(19) United States
(12) Patent Application Publication
(10) Pub. No.: US 2024/0175010 A1
(43) Pub. Date: May 30, 2024

(54) Methods of Library Preparation

(71) Applicant: Illumina, Inc., San Diego, CA (US)

(72) Inventors: Allison Yunghans, San Diego, CA (US); Angelica Marie Barr Schalembier, San Diego, CA (US); Kayla Busby, San Diego, CA (US); Stephen M. Gross, San Diego, CA (US)

(73) Assignee: Illumina, Inc., San Diego, CA (US)

(21) Appl. No.: 18/476,486

(22) Filed: Sep. 28, 2023

Related U.S. Application Data
(63) Continuation of application No. PCT/US2022/022167, filed on Mar. 28, 2022.
(60) Provisional application No. 63/224,201, filed on Jul. 21, 2021.
(60) Provisional application No. 63/167,150, filed on Mar. 29, 2021.

(51) Int. Cl. C12N 15/10; C12N 9/00; C12N 9/22; C12N 9/24; C12Q 1/6874

(52) U.S. Cl. CPC C12N 15/1082; C12N 9/22; C12N 9/2497; C12N 9/93; C12N 15/1096; C12Q 1/6874; C12Y 302/0202; C12Y 302/02023; C12Y 302/02027; C12Y 302/02029; C12Y 605/01001

(57) ABSTRACT

Disclosed herein is a modified transposon end sequence comprising a mosaic end sequence, wherein the mosaic end sequence comprises one or more mutation as compared to a wild-type mosaic end sequence, wherein the mutation comprises a substitution with a uracil, an inosine, a ribose, an 8-oxoguanine, a thymine glycol, a modified purine, or a modified pyrimidine. Also disclosed are transposome complexes comprising these modified transposon end sequences and methods of library preparation using these modified transposon end sequences.

### CROSS-REFERENCE TO RELATED APPLICATION

This application is a bypass continuation of PCT/US2022/022167, filed Mar. 28, 2022, which claims the benefit of priority of U.S. Provisional Application No. 63/167,150, filed Mar. 29, 2021, and U.S. Provisional Application No. 63/224,201, filed Jul. 21, 2021, the contents of which are each incorporated by reference herein in their entireties for any purpose.

### SEQUENCE LISTING

The present application is filed with a Sequence Listing in electronic format. The Sequence Listing is provided as a file entitled “2024-01-04_01243-0027-00US_ST26” created on Jan. 4, 2024, which is 27,170 bytes in size. The information in the electronic format of the sequence listing is incorporated herein by reference in its entirety.

### DESCRIPTION

### Field

This disclosure relates to modified transposon end sequences comprising a mosaic end sequence, wherein the mosaic end sequence comprises one or more mutation as compared to a wild-type mosaic end sequence, wherein the mutation comprises a substitution with a uracil, an inosine, a ribose, 8-oxoguanine, a thymine glycol, a modified purine, or a modified pyrimidine. This disclosure also relates to transposome complexes comprising these modified transposon end sequences and methods of library preparation using these modified transposon end sequences.

### BACKGROUND

Fragmentation of DNA samples is required for NGS, but current methods are limited to (A) mechanical approaches that require expensive capital equipment, (B) enzymatic strategies that have variable performance based on sample concentrations and time, and (C) tagmentation-based approaches that place limitations on library adapter structure.

The first step in preparing libraries for NGS is DNA fragmentation, in which DNA fragments with a size distribution centered around an optimal length are generated, typically in the range of several hundred basepairs. There are a variety of methods for DNA fragmentation which can be classified as either mechanical or enzymatic. Mechanical methods include sonication, acoustic shearing, and nebulization (See Maria S. Poptsova et al., Scientific Reports 4 (2014)). These mechanical methods all require specialized capital equipment and have the potential to introduce DNA damage. In contrast, enzymes do not require specialized equipment, reducing upfront costs for the user. Because of this, users may prefer library preparation products that rely on enzymatic fragmentation.

Beyond transposases, such as those comprised in some Illumina library preparation products, alternative classes of enzymes that can be used for DNA fragmentation include restriction enzymes and nicking enzymes. Restriction enzymes recognize and cut at a specific site, which leads to fragmentation bias, and are thus not commonly employed for NGS applications. In contrast, nicking enzymes introduce random single-stranded cuts in the DNA substrate. An example of a product that enables enzymatic fragmentation based on nicking enzymes is NEBNext Fragmentase. In this product, one enzyme generates random nicks within the substrate DNA, and separate enzyme cuts the complementary strand, resulting in DNA fragmentation. An exemplary protocol using this method would be NEBNext dsDNA Fragmentase (See NEBNext® for DNA Sample Prep for the Illumina Platform, NEB, 2019).

Because the NEB Fragmentase fragments DNA without adding adapter sequences, this workflow is compatible with various existing ligation-based library preparation workflows, including PCR-free approaches. However, these fragmentase enzymes can turn-over several times, making the fragmentation time- and concentration-dependent, and thus optimization of this reaction for the user's specific sample type is often necessary to attain the appropriate fragment size distribution (See Joseph P. Dunham and Maren L. Friesen, Cold Spring Harbor Protocols 9:820-34 (2013)). In contrast, transposase-mediated fragmentation is limited to one turnover based on its dependence on the preloaded transposon substrate, but transposase-mediated fragmentation requires introduction of the mosaic end sequence into the DNA fragments.

In summary, enzymatic fragmentation methods are preferred by many users because they do not require specialized equipment and are more amenable to high-throughput applications. However, present enzymatic fragmentation methods do not have the advantages of BLTs, such as DNA quantification and library normalization with BLTs, thus differentiating BLT-based methods from those using fragmentases.

A critical requirement for transposition of transposon Tn5 is the “mosaic end” (ME) that is specifically recognized by Tn5 transposase and required for its transposition activity. Tn5 transposase natively recognizes the “outside end” (OE) and “inside end” (IE) sequences, which have been shown to be highly intolerant to mutations, with most mutations leading to decreased activity. Later work demonstrated that a chimeric sequence derived from IE and OE, termed the “mosaic end,” (ME) along with a mutant Tn5 enzyme, increased the transposition activity approximately 100-fold relative to the native system. This hyperactive system forms the basis for the Illumina DNA Flex PCR-Free (research use only, RUO) technology, previously known as Illumina's Nextera technology. Crystal structures of Tn5 transposase in complex with DNA substrates indicate that 13 of the 19 basepairs have nucleobase-specific crystal contacts, while other bases have been shown to play a role in catalysis.

Tn5 transposase and bead-linked transposomes (BLTs) are powerful tools that mediate simultaneous enzymatic DNA fragmentation and adapter ligation, or tagmentation, for NGS library preparation. The tagmentation process eliminates requirements for mechanical or enzymatic fragmentation of sample DNA, enzymatic end-repair, and ligation of adapters, resulting in a facile library preparation method. However, a constraint of these systems is the requirement that a single-stranded 19-nucleotide mosaic end sequence be incorporated adjacent to 5′ ends of the library insert. While this can be easily leveraged for standard library preparation, formation of libraries with additional features, such as forked adapters, barcodes, and unique molecular identifiers (UMIs), while retaining compatibility with standard sequencing methods is difficult.

The fragmentase BLT (fBLT) technology described herein overcomes these technical challenges by leveraging the unique advantages of BLTs, while additionally eliminating the constraint of previous tagmentation approaches that requires a defined 19-basepair sequence adjacent to the library insert. By decoupling the enzymatic fragmentation and adapter tagging steps, the addition of features such as forked adapters, barcodes and UMIs can be enabled, while retaining compatibility with standard sequencing methods. Based on these unique advantages, fBLTs could be employed in a variety of applications such as UMI library preparation and PCR-free library preparation.

The modified transposon ends sequences disclosed herein can eliminate the constraint requiring the 19-bp mosaic end sequence adjacent to the library insert and enables hybrid Tn5-ligation library preparation approaches, thus enabling BLTs to be leveraged in library preparation workflows that have been developed based on ligation chemistries. This disclosure describes that Tn5 can tolerate a number of mutations and nucleobase modifications within the mosaic end substrate.

### SUMMARY

In accordance with the description, library preparation methods can comprise transposition by bead-linked transposomes (BLTs), cleavage of modified mosaic end sequences comprised in transposon ends, and adapter ligation.

Embodiment 1. A modified transposon end sequence comprising a mosaic end sequence, wherein the mosaic end sequence comprises one or more mutations as compared to a wild-type mosaic end sequence, wherein the mutation comprises a substitution with
a. a uracil;
b. an inosine;
c. a ribose;
d. an 8-oxoguanine;
e. a thymine glycol;
f. a modified purine; or
g. a modified pyrimidine.

Embodiment 2. A modified transposon end sequence of embodiment 1, wherein the wild-type mosaic end sequence comprises SEQ ID No: 1, and further wherein the one or more mutations comprise a substitution at A16, C17, A18, and/or G19.

Embodiment 3. The modified transposon end sequence of embodiment 1-2, wherein the mosaic end sequence comprises no more than 8 mutations as compared to the wild-type sequence.

Embodiment 4. The modified transposon end sequence of embodiment 2, wherein the mosaic end sequence comprises one or more mutations as compared to SEQ ID NO: 1 in addition to the one or more mutations at A16, C17, A18, and/or G19.

Embodiment 5. The modified transposon end sequence of embodiment 2, wherein the mosaic end sequence comprises from one to four substitution mutations as compared to SEQ ID NO: 1 in addition to the one or more mutations at A16, C17, A18, and/or G19.

Embodiment 6. The modified transposon end of embodiment 2, wherein the mosaic end sequence has one substitution mutation as compared to SEQ ID NO: 1 in addition to the one or more mutations at A16, C17, A18, and/or G19.

Embodiment 7. The modified transposon end of embodiment 2, wherein the mosaic end sequence has two substitution mutations as compared to SEQ ID NO: 1 in addition to the one or more mutations at A16, C17, A18, and/or G19.

Embodiment 8. The modified transposon end of embodiment 2, wherein the mosaic end sequence has three substitution mutations as compared to SEQ ID NO: 1 in addition to the one or more mutations at A16, C17, A18, and/or G19.

Embodiment 9. The modified transposon end of embodiment 2, wherein the mosaic end sequence has four substitution mutations as compared to SEQ ID NO: 1 in addition to the one or more mutations at A16, C17, A18, and/or G19.

Embodiment 10. The modified transposon end sequence of any one of embodiments 2-9, wherein the:
a. the substitution at A16 is A16T, A16C, A16G, A16U, A16Inosine, A16Ribose, A16-8-oxoguanine, A16Thymine glycol, A16Modified purine, or A16 Modified pyrimidine;
b. the substitution at C17 is C17T, C17A, C17G, C17U, C17Inosine, C17Ribose, C17-8-oxoguanine, C17Thymine glycol, C17Modified purine, or C17Modified pyrimidine;
c. the substitution at A18 is A18G, A18T, A18C, A18U, A18Inosine, A18Ribose, A18-8-oxoguanine, A18Thymine glycol, A18Modified purine, or A18Modified pyrimidine; and/or
d. the substitution at G19 is G19T, G19C, G19A, G19U, G19Inosine, G19Ribose, G19-8-oxoguanine, G19Thymine glycol, G19Modified purine, or G19Modified pyrimidine.

Embodiment 11. The modified transposon end sequence of any one of embodiments 2-9, wherein the mutation comprises a substitution with:
a. a uracil;
b. an inosine;
c. a ribose;
d. an 8-oxoguanine
e. a thymine glycol;
f. a modified purine; and/or
g. a modified pyrimidine

Embodiment 12. The modified transposon end sequence of any one of embodiments 2-11, wherein the modified transposon end sequence comprises a mutation at A16, C17, A18, or G19.

Embodiment 13. The modified transposon end sequence of any one of embodiments 2-11, wherein the modified transposon end sequence comprises two mutations chosen from mutations at A16, C17, A18, or G19.

Embodiment 14. The modified transposon end sequence of any one of embodiments 2-11, wherein the modified transposon end sequence comprises three mutations chosen from mutations at A16, C17, A18, or G19.

Embodiment 15. The modified transposon end sequence of any one of embodiments 2-11, wherein the modified transposon end sequence comprises four mutations at A16, C17, A18, and G19.

Embodiment 16. The modified transposon end of any one of embodiments 2-11, wherein the modified transposon end sequence has from one to four substitution mutations as compared to SEQ ID NO: 1 at A16, C17, A18, and/or G19.

Embodiment 17. The modified transposon end of any one of embodiments 1-11, wherein the modified transposon end sequence has one substitution mutation as compared to the wild-type sequence.

Embodiment 18. The modified transposon end of any one of embodiments 1-11, wherein the modified transposon end sequence has two substitution mutations as compared to the wild-type sequence.

Embodiment 19. The modified transposon end of any one of embodiments 1-11, wherein the modified transposon end sequence has three substitution mutations as compared to the wild-type sequence.

Embodiment 20. The modified transposon end of any one of embodiments 1-11, wherein the modified transposon end sequence has four substitution mutations as compared to the wild-type sequence.

Embodiment 21. The modified transposon end of any one embodiments 1-20, wherein the modified purine is 3-methyladenine or 7-methylguanine.

Embodiment 22. The modified transposon end of any one embodiments 1-20, wherein the modified pyrimidine is 5-methylcytosine, 5-formylcytosine, or 5-carboxycytosine.

Embodiment 23. A transposome complex comprising:
a. a transposase;
b. a first transposon comprising a modified transposon end sequence comprising a uracil, an inosine, a ribose, an 8-oxoguanine, a thymine glycol, a modified purine, and/or a modified pyrimidine; and
c. a second transposon comprising a second transposon end sequence complementary to at least a portion of the first transposon end sequence.

Embodiment 24. The transposome complex of embodiment 23, wherein the first transposon comprises a ribose, a uracil, an inosine, an 8-oxoguanine, a thymine glycol, a modified purine, and/or a modified pyrimidine and the transposome complex is in solution.

Embodiment 25. The transposome complex of embodiment 23, wherein the first transposon comprises a uracil, an inosine, an 8-oxoguanine, a thymine glycol, a modified purine, and/or a modified pyrimidine and the transposome complex is immobilized on a solid support.

Embodiment 26. The transposome complex of any one of embodiments 23-25, wherein the first transposon comprises a modified transposon end sequence of any one of embodiments 1-22.

Embodiment 27. The transposome complex of any one of embodiments 23-26, wherein the transposase is Tn5.

Embodiment 28. The transposome complex of any one of embodiments 23-27, wherein the first transposon is the transferred strand.

Embodiment 29. The transposome complex of any one of embodiments 23-28, wherein the second transposon is the non-transferred strand.

Embodiment 30. The transposome complex of any one of embodiments 23-29, wherein a uracil in the first transposon is base paired with an A in the second transposon.

Embodiment 31. The transposome complex of any one of embodiments 23-30, wherein an inosine in the first transposon is base paired with a C in the second transposon.

Embodiment 32. The transposome complex of any one of embodiments 23-31, wherein a ribose in the first transposon is base paired with an A, C, T, or G in the second transposon.

Embodiment 33. The transposome complex of any one of embodiments 23-32, wherein a thymine glycol in the first transposon is base paired with an A in the second transposon.

Embodiment 34. The transposome complex of any one of embodiments 23-33, wherein a modified purine is a 3-methyladenine in the first transposon that is base paired with an T in the second transposon.

Embodiment 35. The transposome complex of any one of embodiments 23-34, wherein a modified purine is a 7-methylguanine in the first transposon that is base paired with an C in the second transposon.

Embodiment 36. The transposome complex of any one embodiments 23-34, wherein the modified pyrimidine is 5-methylcytosine, 5-formylcytosine, or 5-carboxycytosine in the first transposon that is base paired with a G in the second transposon.

Embodiment 37. The transposome complex of any one of embodiments 23-36, wherein the first or second transposon comprises an affinity element.

Embodiment 38. The transposome complex of embodiment 37, wherein the first transposon comprises an affinity element.

Embodiment 39. The transposome complex of embodiment 38, wherein the affinity element is attached to the 5′ end of the first transposon.

Embodiment 40. The transposome complex of embodiment 38 or 39, wherein the first transposon comprised in the targeted transposome complex comprises a linker.

Embodiment 41. The transposome complex of embodiment 40, wherein the linker has a first end attached to the 5′ end of the first transposon and a second end attached to an affinity element.

Embodiment 42. The transposome complex of embodiment 37, wherein the second transposon comprises an affinity element.

Embodiment 43. The transposome complex of embodiment 42, wherein the affinity element is attached to the 3′ end of the second transposon.

Embodiment 44. The transposome complex of embodiment 43, wherein the second transposon comprises SEQ ID NO: 13.

Embodiment 45. The transposome complex of embodiment 44, wherein the second transposon comprises a linker.

Embodiment 46. The transposome complex of embodiment 45, wherein the linker has a first end attached to the 3′ end of the second transposon and a second end attached to an affinity element.

Embodiment 47. The transposome complex of any one of embodiments 37-46, wherein the affinity element comprises biotin, avidin, streptavidin, an antibody, or an oligonucleotide.

Embodiment 48. The transposome complex of any one of embodiment 23-47, wherein the second transposon comprises:
a. a second transposon end sequence complementary to SEQ ID NO: 1; or
b. a second transposon end fully complementary to the first transposon end.

Embodiment 49. The transposome complex of embodiment 48, wherein the first transposon comprises a modified transposon end sequence comprising an A16U, A16-8-oxoguanine, or A16Inosine substitution as compared to SEQ ID NO: 1 and the second transposon comprises a second transposon end sequence complementary to SEQ ID NO: 1 or a second transposon end fully complementary to the first transposon end.

Embodiment 50. The transposome complex of embodiment 48, wherein the first transposon comprises a modified transposon end sequence comprising an C17-8-oxoguanine or C17Inosine substitution as compared to SEQ ID NO: 1 and the second transposon comprises a second transposon end sequence complementary to SEQ ID NO: 1 or a second transposon end fully complementary to the first transposon end.

Embodiment 51. The transposome complex of embodiment 48, wherein the first transposon comprises a modified transposon end sequence comprising an A18-8-oxoguanine or A18Inosine substitution as compared to SEQ ID NO: 1 and the second transposon comprises a second transposon end sequence complementary to SEQ ID NO: 1 or a second transposon end fully complementary to the first transposon end.

Embodiment 52. The transposome complex of embodiment 48, wherein the first transposon comprises a modified transposon end sequence comprising an G19-8-oxoguanine or G19Inosine substitution as compared to SEQ ID NO: 1 and the second transposon comprises a second transposon end sequence complementary to SEQ ID NO: 1 or a second transposon end fully complementary to the first transposon end.

Embodiment 53. The transposome complexes of any one of embodiments 23-52, wherein the transposome complexes are in solution.

Embodiment 54. A solid support having transposome complexes of any one of embodiments 23-52 immobilized thereon.

Embodiment 55. A method of fragmenting a double-stranded nucleic acid comprising combining a sample comprising double-stranded nucleic acid with the transposome complexes of any one of embodiments 23-53 or the solid support of embodiment 54 and preparing fragments.

Embodiment 56. A method of preparing double-stranded nucleic acid fragments that lack all or part of the first transposon end comprising:
a. combining a sample comprising nucleic acid with the transposome complexes of any one of embodiments 23-53 or with the solid support of embodiment 54 and preparing fragments; and
b. combining the sample with (1) an endonuclease or (2) a combination of a DNA glycosylase and heat, basic conditions, or an endonuclease/lyase that recognizes abasic sites, and cleaving the first transposon end at the uracil, inosine, ribose, 8-oxoguanine, thymine glycol, modified purine, and/or a modified pyrimidine within the mosaic sequence to remove all or part of the first transposon end from the fragments.

Embodiment 57. The method of embodiment 56, wherein the modified purine is 3-methyladenine or 7-methylguanine.

Embodiment 58. The method of embodiment 56, wherein the modified pyrimidine is 5-methylcytosine, 5-formylcytosine, or 5-carboxycytosine.

Embodiment 59. The method of embodiment 57 or 58, further comprising sequencing the fragments after removing all or part of the first transposon end from the fragment.

Embodiment 60. The method of embodiment 59, wherein the method does not require amplification of fragments before sequencing.

Embodiment 61. The method of embodiment 59, wherein fragments are amplified before sequencing.

Embodiment 62. The method of any one of embodiments 59-61, further comprising enriching fragments of interest after ligating the adapter and before sequencing.

Embodiment 63. A method of preparing double-stranded nucleic acid fragments comprising adapters comprising:
a. combining a sample comprising nucleic acid with the transposome complexes of any one of embodiments 23-53 or with the solid support of embodiment 54 and preparing fragments;
b. combining the sample with (1) an endonuclease or (2) a combination of a DNA glycosylase and heat, basic conditions, or an endonuclease/lyase that recognizes abasic sites and cleaving the first transposon end at the uracil, inosine, ribose, 8-oxoguanine, thymine glycol, modified purine, and/or modified pyrimidine within the mosaic end sequence to remove all or part of the first transposon end from the fragments; and
c. ligating an adapter onto the 5′ and/or 3′ ends of the fragments.

Embodiment 64. The method of embodiment 63, wherein the modified purine is 3-methyladenine or 7-methylguanine.

Embodiment 65. The method of embodiment 63, wherein the modified pyrimidine is 5-methylcytosine, 5-formylcytosine, or 5-carboxycytosine.

Embodiment 66. The method of any one of embodiments 56-65, wherein the nucleic acid is double-stranded DNA.

Embodiment 67. The method of any one of embodiments 56-65, wherein the nucleic acid is RNA, and double-stranded cDNA or DNA:RNA duplexes are generated before combining with the transposome complexes.

Embodiment 68. The method of any one of embodiments 56-67, wherein the all or part of the first transposon end that is cleaved is partitioned away from the rest of the sample.

Embodiment 69. The method of any one of embodiments 63-68, further comprising filling in the 3′ ends of the fragments and phosphorylating the 3′ ends of fragments with a kinase before ligating.

Embodiment 70. The method of embodiment 69, wherein the filling in is performed with T4 DNA polymerase.

Embodiment 71. The method of embodiment 70, further comprising adding a single A overhang to the 3′ end of the fragments.

Embodiment 72. The method of embodiment 71, wherein a polymerase adds the single A overhang.

Embodiment 73. The method of embodiment 72, wherein the polymerase is (i) Taq or (ii) Klenow fragment, exo-.

Embodiment 74. The method of any one of embodiments 56-73, wherein the fragments comprise 0-3 bases of the mosaic end sequence.

Embodiment 75. The method of any one of embodiments 56-74, wherein preparing fragments leads to preparation of at least 50%, at least 60%, at least 70%, at least 80%, or at least 90% the number of fragments, as compared with preparing fragments with a transposome complex that comprises a first transposon comprising a transposon end sequence comprising a wildtype mosaic end sequence comprising SEQ ID No: 1.

Embodiment 76. The method of any one of embodiments 63-75, further comprising sequencing the fragments after ligating the adapter.

Embodiment 77. The method of embodiment 76, wherein the method does not require amplification of fragments before sequencing.

Embodiment 78. The method of embodiment 77, wherein fragments are amplified before sequencing.

Embodiment 79. The method of any one of embodiments 76-78, further comprising enriching fragments of interest after ligating the adapter and before sequencing.

Embodiment 80. The method of any one of embodiments 56-79, wherein the modified transposon end sequence comprises a uracil and the combination of a DNA glycosylase and an endonuclease/lyase that recognizes abasic sites is a uracil-specific excision reagent (USER).

Embodiment 81. The method of embodiment 80, wherein the USER is a mixture of uracil DNA glycosylase and endonuclease VIII or endonuclease III.

Embodiment 82. The method of any one of embodiments 56-79, wherein the modified transposon end sequence comprises an inosine and the endonuclease is endonuclease V.

Embodiment 83. The method of any one of embodiments 56-79, wherein the modified transposon end sequence comprises a ribose and the endonuclease is RNAse HII.

Embodiment 84. The method of any one of embodiments 56-79, wherein the modified transposon end sequence comprises a 8-oxoguanine and the endonuclease is formamidopyrimidine-DNA glycosylase (FPG) or oxoguanine glycosylase (OGG).

Embodiment 85. The method of any one of embodiments 56-79, wherein the modified transposon end sequence comprises a thymine glycol and the DNA glycosylase is endonuclease EndoIII (Nth) or Endo VIII.

Embodiment 86. The method of any one of embodiments 56-79, wherein the modified transposon end sequence comprises a modified purine and the DNA glycosylase is human 3-alkyladenine DNA glycosylase and the endonuclease is endonuclease III or VIII.

Embodiment 87. The method of embodiment 86, wherein the modified purine is 3-methyladenine or 7-methylguanine.

Embodiment 88. The method of any one of embodiments 56-79, wherein the modified transposon end sequence comprises a modified pyrimidine and (1) the DNA glycosylase is thymine-DNA glycosylase (TDG) or mammalian DNA glycosylase-methyl-CpG binding domain protein 4 (MBD4) and the endonuclease/lyase that recognizes abasic sites is the endonuclease is endonuclease III or VIII; or (2) the endonuclease is DNA glycosylase/lyase ROS1 (ROS1).

Embodiment 89. The method of embodiment 88, wherein the modified pyrimidine is 5-methylcytosine, 5-formylcytosine, or 5-carboxycytosine.

Embodiment 90. The method of any one of embodiments 56-89, wherein the first transposon comprises a modified transposon end sequence comprising more than one mutation chosen from a uracil, an inosine, a ribose, 8-oxoguanine, a thymine glycol, a modified purine, or a modified pyrimidine and the (1) an endonuclease or (2) a combination of a DNA glycosylase and heat, basic conditions, or an endonuclease/lyase that recognizes abasic sites is an enzyme mixture.

Embodiment 91. The method of embodiment 90, wherein the modified purine is 3-methyladenine or 7-methylguanine.

Embodiment 92. The method of embodiment 90, wherein the modified pyrimidine is 5-methylcytosine, 5-formylcytosine, or 5-carboxycytosine.

Embodiment 93. The method of any one of embodiments 63-92, wherein cleaving the first transposon end generates a sticky end for ligating the adapter.

Embodiment 94. The method of embodiment 93, wherein the sticky end is longer than one base.

Embodiment 95. The method of any one of embodiments 63-94, wherein the adapter comprises a double-stranded adapter.

Embodiment 96. The method of any one of embodiments 63-95, wherein adapters are added to the 5′ and 3′ end of fragments.

Embodiment 97. The method of embodiment 96, wherein the adapters added to the 5′ and 3′ end of the fragments are different.

Embodiment 98. The method of any one of embodiments 63-97, wherein the adapter comprises a unique molecular identifier (UMI), primer sequence, anchor sequence, universal sequence, spacer region, index sequence, capture sequence, barcode sequence, cleavage sequence, sequencing-related sequence, and combinations thereof.

Embodiment 99. The method of any one of embodiments 98, wherein the adapter comprises a UMI.

Embodiment 100. The method of embodiment 99, wherein an adapter comprising a UMI is ligated to both the 3′ and 5′ end of fragments.

Embodiment 101. The method of any one of embodiments 63-100, wherein the adapter is a forked adapter.

Embodiment 102. The method of any one of embodiments 63-101, wherein the ligating is performed with a DNA ligase.

Embodiment 103. The method of any one of embodiments 63-102, wherein the method is performed in a single reaction vessel.

Embodiment 104. The method of any one of embodiments 56-103, wherein the density of transposomes immobilized on the solid surface is selected to modulate fragment size and library yield of the immobilized fragments.

Embodiment 105. The method of any one of embodiments 56-104, wherein the method allows for bead-based normalization.

Embodiment 106. The method of any one of embodiments 56-105, wherein the sample comprises partially fragmented DNA.

Embodiment 107. The method of any one of embodiments 56-106, wherein the sample is formalin fixed paraffin embedded tissue or cell-free DNA.

Embodiment 108. The method of any one of embodiments 56-107, wherein the library comprises fragments prepared by a single tagmentation event.

Embodiment 109. A pair of transposons having a first transposon and a second transposon, wherein the first transposon comprises a modified transposon end sequence of any one of embodiments 1-22 and wherein the second transposon comprises:
a. a transposon end sequence comprising a mosaic end sequence complementary to the wild-type mosaic end sequence; or
b. a transposon end sequence fully complementary to the first transposon end.

Embodiment 110. The pair of transposons of embodiment 109, wherein the first transposon comprises a modified transposon end sequence comprising an A16U, A16-8-oxoguanine, or A16Inosine substitution as compared to SEQ ID NO: 1 and the second transposon comprises a second transposon end sequence complementary to SEQ ID NO: 1 or a second transposon end fully complementary to the first transposon end.

Embodiment 111. The pair of transposons of embodiment 109, wherein the first transposon comprises a modified transposon end sequence comprising an C17-8-oxoguanine or C17Inosine substitution as compared to SEQ ID NO: 1 and the second transposon comprises a second transposon end sequence complementary to SEQ ID NO: 1 or a second transposon end fully complementary to the first transposon end.

Embodiment 112. The pair of transposons of embodiment 109, wherein the first transposon comprises a modified transposon end sequence comprising an A18-8-oxoguanine or A18Inosine substitution as compared to SEQ ID NO: 1 and the second transposon comprises a second transposon end sequence complementary to SEQ ID NO: 1 or a second transposon end fully complementary to the first transposon end.

Embodiment 113. The pair of transposons of embodiment 109, wherein the first transposon comprises a modified transposon end sequence comprising an G19-8-oxoguanine or G19Inosine substitution as compared to SEQ ID NO: 1 and the second transposon comprises a second transposon end sequence complementary to SEQ ID NO: 1 or a second transposon end fully complementary to the first transposon end.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A and 1B shows an overview of fragmentation methods. (A) The present fragmentation-Tn5 approach uses modification of Tn5-Mosaic End substrate to enable selective cleavage of the mosaic end and subsequent adapter ligation. (B) Standard competing workflow in which input DNA is mechanically sheared or enzymatically fragmented with subsequent end repair and adapter ligation. In both FIGS. 1A and 1B, attachment of Y-shaped adapters containing all standard adapter sequences for Illumina sequencing (P5-i5-A14-ME and ME′-B15′-i7′-P7′) is shown. In an alternate configuration, a short Y-shaped adapter containing only A14-ME and ME′-B15′ can be used, and additional adapter sequences can be added by PCR in a method such as that described in FIG. 2 of US Patent Publication No. 20180201992A1, which is incorporated by reference herein in its entirety.

FIG. 2 outlines the mechanism of Tn5 transposase in standard tagmentation library preparation. The Tn5 transposase enzyme is pre-loaded with a transposon DNA substrate consisting of the cognate “mosaic end” and appended adapter sequences (such as A14 and B15 for Illumina methods). During tagmentation, these transposomes act on genomic DNA, leading to simultaneous fragmentation and tagging with adapter sequences. The A14 and B15 sequences are SEQ ID Nos: 11 and 12, respectively. The ME sequence and its complement (ME′) are SEQ ID Nos: 1 and 4, respectively.

FIG. 3 outlines how bead-linked transposomes (BLTs) enable normalization-free workflow. By conjugating transposomes to a magnetic bead, the amount of DNA that is converted to library is normalized. Additionally, some control of library fragment size is attained through selection of the transposome density. Libraries may also be subjected to Solid Phase Reversible Immobilization (SPRI)-based size selection to gain further control of fragment size. gDNA=genomic DNA.

FIG. 4 outlines enzymatic fragmentation with fragmentase. In the method shown, Enzyme 1 introduces random nicks into one strand, and enzyme 2 introduces cuts opposite from the nick and produces dsDNA breaks. The resulting DNA fragments typically have 1-4 base overhangs on the 5′ end. An exemplary protocol using this method would be NEBNext dsDNA Fragmentase (See NEBNext® for DNA Sample Prep for the Illumina Platform, NEB, 2019 and NEBNext dsDNA Fragmentase Product details available at www.nebj.jp/products/detail/1020.com, Accessed on Mar. 17, 2021).

FIG. 5 shows potential mechanisms for removal of the mosaic end sequence. Possible enzymatic strategies include the use of restriction enzymes, single stranded DNAses, or DNA repair enzymes. In some embodiments, DNA repair enzymes are attractive for their specificity.

FIGS. 6A and 6B show analysis of Tn5v3 activity with mutated mosaic end sequences. (A) Canonical substitutions at various positions are reported based on the transferred strand sequence (SEQ ID NO: 1), with corresponding substitution in the non-transferred strand (SEQ ID NO: 4), except bases noted * indicating the substitution was made only in the transferred strand and a wild-type non-transferred strand was annealed. At position 16A, substitutions were made to T, C, G. At position 17C, substitutions were made to T, A, and G. At position 18A, substitutions were made to G, T, and C. At position 19G, substitutions were made to T, C, and A. Other substitutions to SEQ ID NO: 1 were made as marked. (B) Activity of Tn5v3 transposomes prepared with DNA modifications in the TS. Uracil was basepaired with A and inosine was basepaired with C. Sequences shown are transferred strand, TS (SEQ ID NO: 1) and non-transferred strand, NTS (SEQ ID NO: 4). AU=arbitrary unit.

FIGS. 7A-7C show library preparation using Tn5-fragmentase approach. (A) Diagram of the workflow employed to prepare libraries. (B) Diagram of how a modification-specific endonuclease can cleave a modified base in a 1-step reaction or a modification-specific glycosylase followed by an AP lyase/endonuclease or heat can cleave a modified base in a 2-step reaction. (C) Electropherograms of libraries prepared with DNA modifications. Libraries were treated with either USER, Endonuclease V, or RNAse HII according to manufacturer's protocols (NEB). In this experiment, a large amount of adapter dimer (peak at ˜160 bp) was observed, likely due to non-optimal ligation adapter concentration. ATL=A tailing. LIG=ligation.

FIGS. 8A and 8B show comparison of uracil modification site within ME. (A) Electropherograms of libraries generated with alternative mosaic ends. (B) Qubit yields of libraries with alternative MEs. USER incubation times of 20 and 60 minutes were tested.

FIG. 9 summarizes that fragmentase libraries show the expected ME “scar” adjacent to the library insert. Because of the variable UMI length, some libraries are shifted by 1 bp. The ME scar for each modification site is present as expected. The A16U transferred strand sequence and T16A non-transferred strand sequence are SEQ ID NOs: 5 and 6, respectively. The C17U transferred strand sequence and G17A non-transferred strand sequence are SEQ ID NOs: 7 and 8, respectively. The A18U transferred strand sequence and the T18A non-transferred strand sequence are SEQ ID NOs: 9 and 10, respectively.

FIGS. 10A-10C shows a representative fBLT library preparation. (A) Workflow used in this study. (B) Library yields of enrichment BLT (eBLT) and fragmentase (fBLT) library preparations. (C) Representative Bioanalyzer traces of eBLT and fBLT libraries. Additional workflows with fBLTs will be disclosed herein.

FIG. 11 shows an overview of a fBLT, with a representative modified transposon end comprising a transferred strand with a G19I mutation (SEQ ID NO: 14) and a biotinylated non-transferred strand (SEQ ID NO: 13) that can be used to immobilize the transposomes. B=biotin.

FIG. 12 shows results with fBLTs with different modified bases in the mosaic end (ME sequence) of the first transposon. 16-19 represent positions of modifications from SEQ ID NO: 1. oxoG=oxoguanine; AU=activity unit.

FIGS. 13A-13C show results with different types of fBLTs. (A) Results on percentage conversion with A18Inosine (I18), C17-8-Oxoguanine (O17), and G19U (U19) mutations. (B) Results on variant calling performance with I18, O17, and U19. (C) Results on percentage conversion with I18, G19I (I19), O17, A18O (O18), and G19O (O19). Results indicated generally high performance of BLTs with mosaic ends substituted with inosine with highest performance of G19I (I19).

FIG. 14 presents data on chimeric reads. Use of modified transposon ends comprising uracil may lead to a higher percentage of chimeric reads as compared to modified transposon ends comprising inosine or oxoguanine.

FIGS. 15A and 15B present a comparison of fBLTs versus other library preparation fragmentation methods (i.e., NEBNext® dsDNA Fragmentase® or sonication performed with a Covaris Ultrasonicator following standard procedures). (A) Outline of workflows. (B) Summary of sensitivity and specificity of variant calling performance with different methods measuring a 50 ng input gDNA 1% mixture of NA12877 into a NA12878 background (with 84 heterozygous variants and 0.5% variant allele frequency (VAF)).

FIG. 16 shows error rates for different methods of library preparation with fragmentation, including the substantially higher error rates for samples prepared by sonication.

FIG. 17 shows library conversion efficiency of different fragmentation methods. Overall, fBLTs outperformed the other methods of library conversion. Sample 1 is a genomic DNA 1% mixture of NA12877 in NA12878 background. Samples 2-6 are formalin fixed paraffin embedded (FFPE) tissue. dCq is a measure of DNA quality, with elevated values corresponding to a lower quality sample. Accordingly, the higher conversion efficiency Sample 1 versus the other samples highlights the fact that library conversion is generally reduced from FFPE tissue due to lower DNA quality of FFPE tissue.

FIGS. 18A and 18B summarize a method comprising a single tagmentation event with a fBLT to generate fragments from a sample of FFPE tissue. (A) Outline of workflow. (B) Percentages of fragments rescued from different tissues. Sample numbers are the same as outlined for FIG. 17. dCq is a measure of DNA quality, with elevated values corresponding to a lower quality sample. Thus, the lower percentage of fragments rescued for Sample 1 is indicative of the higher quality in this genomic DNA sample (i.e., there were fewer fragments from single tagmentation events and most fragments were from two tagmentation events) as compared to Samples 2-6 with FFPE tissue. A higher proportion of samples were rescued from FFPE tissue as opposed to genomic DNA, because there can be more single tagmentation events in the FFPE tissue due to its lower quality.

FIG. 19 summarizes some advantages and flexibility of tagmentation protocols using fBLTs. In particular, the method allows for ligation of adapters that allow for different workflows that a user may wish to pursue. As shown, adapters may comprise unique molecular identifiers (UMI) for determining different unique fragments from amplicons of the same fragment. Alternatively, forked adapters may be used in workflows with PCR to incorporate indexes or indexed forked adapters may be used in PCR-free workflows.

FIG. 20 outlines standard workflows for library preparation using fBLTs and optional enrichment. Boxes and triangles refer to steps where a user would have to handle the reaction samples. The overall library preparation time of approximately 5.5 hours is similar to other ligation-based library preparation methods. Optional enrichment may be used, for example, to enrich with a cancer-related panel when preparing a library from a FFPE tissue sample from a cancer patient.

### DESCRIPTION OF THE SEQUENCES

Table 1 below provides a listing of certain sequences referenced herein. Within the table, /3BiotinN/ and /5Phos/ refer to 3′ biotin and 5′ phosphate, respectively. /i8oxodG/ refers to internal 8-oxoG nucleotide, and /38oxodG/ refers to an 8-oxoG nucleotide at the 3′ position.

TABLE 1
Description of the Sequences

| | | SEQ ID |
|---|---|---|
| Description | Sequences | NO |
| Mosaic end (ME) sequence | AGATGTGTATAAGAGACAG | 1 |
| (transferred strand) | | |
| Outside end (OE) | CTGACTCTTATACACAAGT | 2 |
| Inside end (IE) | CTGTCTCTTGATCAGATCT | 3 |
| Mosaic end (ME′) (non- | CTGTCTCTTATACACATCT | 4 |
| transferred strand) | | |
| U16 transferred strand | AGATGTGTATAAGAGUCAG | 5 |
| (TS), Modified ME with | | |
| A16U substitution | | |
| (transferred strand, | | |
| substitution in bold) | | |
| Modified ME′ (non- | TCTACACATATTCTCAGTC | 6 |
| transferred strand) | | |
| presented in 3′-5′ | | |
| orientation) with T16A | | |
| substitution (in bold) | | |
| U17 TS, Modified ME with | AGATGTGTATAAGAGAUAG | 7 |
| C17U substitution | | |
| (transferred strand, | | |
| substitution in bold) | | |
| Modified ME′ (non- | TCTACACATATTCTCTATC | 8 |
| transferred strand, | | |
| presented in 3′-5′ | | |
| orientation) with G17A | | |
| substitution (in bold) | | |
| U18 TS, Modified ME with | AGATGTGTATAAGAGACUG | 9 |
| A18U substitution | | |
| (transferred strand, | | |
| substitution in bold) | | |
| Modified ME′ (non- | TCTACACATATTCTCTGAC | 10 |
| transferred strand, | | |
| presented in 3′-5′ | | |
| orientation) with T18A | | |
| substitution (in bold) | | |
| A14 | TCGTCGGCAGCGTC | 11 |
| B15 | GTCTCGTGGGCTCGG | 12 |
| Biotinylated ME′ (non- | /5Phos/CTGTCTCTTATACACATCT/3BiotinN/ | 13 |
| transferred strand) | | |
| I19 TS, Modified ME with | AGATGTGTATAAGAGACAI | 14 |
| G19I substitution | | |
| (transferred strand, | | |
| substitution in bold) | | |
| U19 TS Modified ME with | AGATGTGTATAAGAGACAU | 15 |
| G19U substitution | | |
| (transferred strand, | | |
| substitution in bold) | | |
| O16 TS, Modified ME with | AGATGTGTATAAGAG/i8oxodG/CAG | 16 |
| A16O substitution | | |
| (transferred strand, | | |
| substitution in bold) | | |
| O17 TS, Modified ME with | AGATGTGTATAAGAGA/i8oxodG/AG | 17 |
| C17O substitution | | |
| (transferred strand, | | |
| substitution in bold) | | |
| O18 TS, Modified ME with | AGATGTGTATAAGAGAC/i8oxodG/G | 18 |
| A18O substitution | | |
| (transferred strand, | | |
| substitution in bold) | | |
| O19 TS Modified ME with | AGATGTGTATAAGAGACA/38oxodG/ | 19 |
| G19O substitution | | |
| (transferred strand, | | |
| substitution in bold) | | |
| I16 TS, Modified ME with | AGATGTGTATAAGAGICAG | 20 |
| A16I substitution | | |
| (transferred strand, | | |
| substitution in bold) | | |
| I17 TS, Modified ME with | AGATGTGTATAAGAGAIAG | 21 |
| C17I substitution | | |
| (transferred strand, | | |
| substitution in bold) | | |
| I18 TS, Modified ME with | AGATGTGTATAAGAGACIG | 22 |
| A18I substitution | | |
| (transferred strand, | | |
| substitution in bold) | | |

### DESCRIPTION OF THE EMBODIMENTS

### I. Modified Transposon Ends With Mutations in the Mosaic End Sequence

Described herein are modified transposon end sequences comprising a mosaic end sequences. In some embodiments, these modified transposon end sequences comprise a mosaic end sequence that allows for cleavage and removal of the mosaic end sequence after transposition. A critical requirement for transposition is the “mosaic end” (ME) which is specifically recognized by Tn5 and required for its transposition activity. Tn5 natively recognizes the “outside end” (OE) and “inside end” (IE) sequences (as shown in Table 2), which have been shown to be highly intolerant to mutations, with most mutations leading to decreased activity (See J. C. Makris et al. PNAS 85(7):2224-28 (1988)). Later work demonstrated that a chimeric sequence derived from IE and OE, termed the “mosaic end” (Table 2), along with a mutant Tn5 enzyme, increased the transposition activity approximately 100-fold relative to the native system (See Maggie Zhou et al., Journal of Molecular Biology 276(5): 913-25 (1998)). This hyperactive system is used in Illumina's Illumina DNA Flex PCR-Free (RUO) products. Crystal structures of Tn5 in complex with DNA substrates indicate that 13 of the 19 basepairs have nucleobase-specific crystal contacts (See Douglas R. Davies et al., Science 289 5476:77-85 (2000)), while other bases have been shown to play a role in catalysis (See Mindy Steiniger-White et al., Journal of Molecular Biology 322(5): 971-82 (2002)). Typically, activity of Tn5 has been assessed by in vivo reporter systems (papillation assays, described in Zhou et al. J. Mol. Biol. 276:913-925 (1998)).

TABLE 2
Known DNA substrates of Tn5 transposase

| Substrate | Sequence | SEQ ID NO |
|---|---|---|
| Outside End | CTG [symbol] CTCTT [symbol] CA [symbol] T | 2 |
| (OE) | | |
| Inside End | CTGTCTCTTGATCAGATCT | 3 |
| (IE) | | |
| Mosaic End | CTGTCTCTT [symbol] CA [symbol] T | 4 |
| (ME) | | |

In Table 2, sequences in normal font indicate shared sequences, sequences in italics with double-underline are derived from the native OE substrate, and sequences in bold italics are derived from the native IE substrate.

A representative wild-type mosaic end sequence (transferred strand) is SEQ ID NO: 1. A variety of mutant Tn5 and transposon ends are described in WO 2015160895 and U.S. Pat. No. 9,080,211, each of which are incorporated by reference in their entirety herein, and may be appropriate for use in the methods described herein.

Several DNA enzymes or enzyme combinations can mediate the selective removal of modified bases such as uracil, inosine, ribose bases, 8-oxo G, thymine glycol, modified purines, and modified pyrimidines among others (See Table 3 and Properties of DNA Repair Enzymes and Structure-specific Endonucleases, New England Biolabs, downloaded Jan. 20, 2022, from www.international.neb.com/tools-and-resources/selection-charts and Jacobs and Schär Chromosoma 121:1-20 (2012)). Such enzymes include modification-specific endonucleases or modification-specific glycosylases. Modified purines for use with modification-specific glycosylases include 3-methyladenine (3mA) and 7-methylguanine (7mG). Modified pyrimidines for use with modification specific-glycosylases may include 5-methylcytosine (5mC), 5-formylcytosine (5fC), and 5-carboxycytosine (5caC). Selective removal of uracil and 8-oxoG using DNA repair enzymes are already used in certain sequencing platforms.

Because only one strand of the mosaic end, called the “transferred strand” is covalently appended to the library insert during transposition, incorporation of such a modified base, specifically into the mosaic end transferred strand, could enable selective cleavage and removal of the mosaic end transferred strand. However, this type of mosaic end cleavage and removal would require mutation of the mosaic end sequence from its canonical sequence (SEQ ID NO: 1).

TABLE 3
Examples of base modifications and enzymatic strategies for fBLT

| | Possible | Possible |
|---|---|---|
| Base | modification-specific | modification-specific |
| modification | N-glycosylases* | endonucleases |
| Uracil | UNG/UDG | |
| Inosine | | Endo V |
| Ribose base | | RNAse HII |
| 8-oxoguanine | | Fpg, OGG |
| Thymine glycol | EndoIII (Nth), Endo VIII | |
| Modified | hAAG | |
| purines (e.g., | | |
| 3 mA and 7 mG) | | |
| Modified | TDG, MBD4 | ROS1 |
| pyrimidines | | |
| (e.g., mC, fC, | | |
| caC) | | |

*N-glycosylases can be paired with an AP lyase/endonuclease (e.g., EndoIII or EndoVIII). As an alternative, abasic sites are chemically labile and may be cleaved with heat and/or basic conditions.

In Table 3, Endo=endonuclease, FPG=formamidopyrimidine-DNA glycosylase, OGG=oxoguanine glycosylase (OGG), hAAG=Human 3-alkyladenine DNA glycosylase, UNG=uracil-N-glycosylase, Nth=cloned nth gene, TDG=thymine-DNA glycosylase, MBD4=mammalian DNA glycosylase-methyl-CpG binding domain protein 4, and ROS1=endonuclease ROS1 (with bifunctional DNA glycosylase/lyase activity).

Disclosed herein is a modified transposon end sequence comprising a mosaic end sequence, wherein the mosaic end sequence comprises one or more mutation as compared to a wild-type mosaic end sequence, wherein the mutation comprises a substitution with a uracil; an inosine; a ribose; an 8-oxoguanine; a thymine glycol; a modified purine (such as 3mA or 7mG); or a modified pyrimidine. In some embodiments, these substitutions are used in methods to cleave the transposon end after transposition, as described below.

In some embodiments, the mosaic end sequence may be a mosaic end sequence for use with a Tn5 transposase. In some embodiments, a modified transposon end sequence has mutations in a mosaic end sequence as compared to SEQ ID NO: 1.

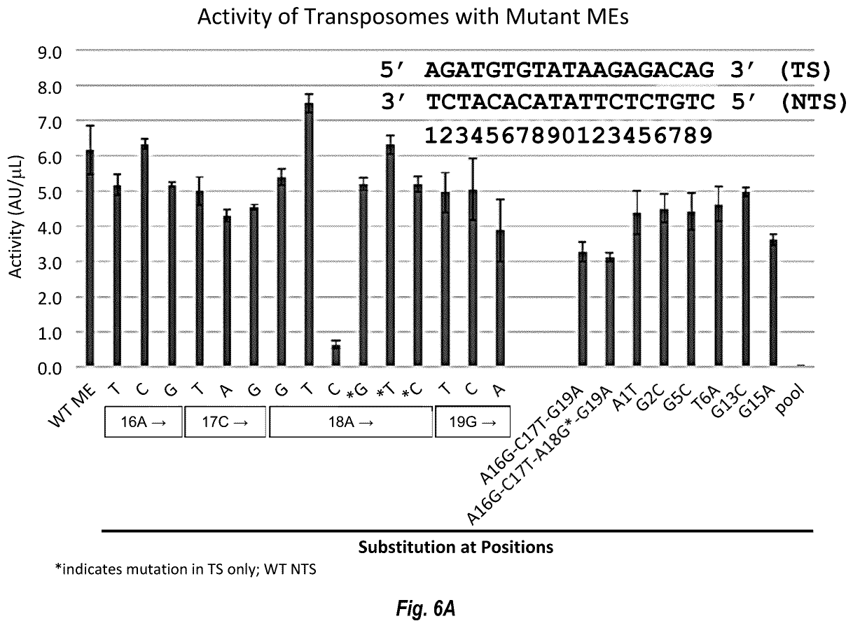

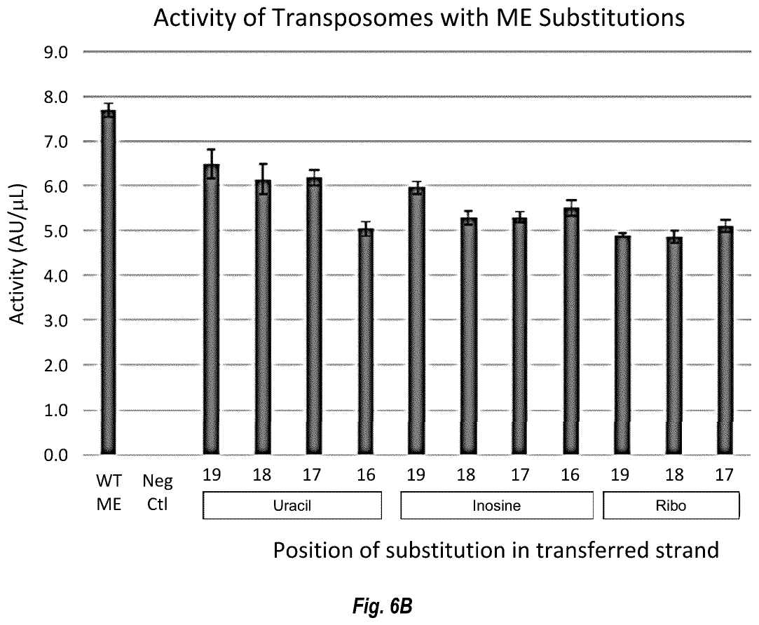

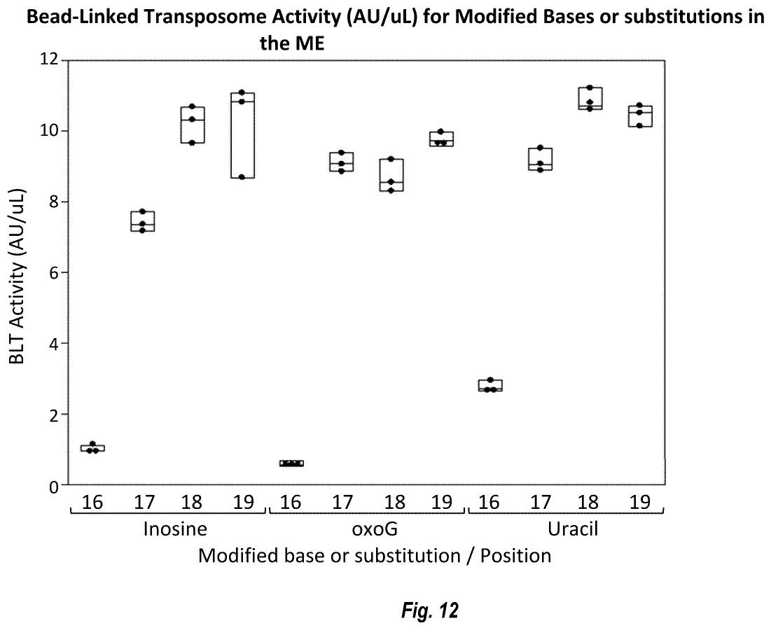

In some embodiments, a modified transposon end sequence comprises a mosaic end sequence comprising one or more mutation as compared to SEQ ID No: 1, wherein the one or more mutations comprise a substitution at A16, C17, A18, and/or G19. In some embodiments, a modified transposon end sequence comprises a mosaic end sequence comprising a substitution at A16. In some embodiments, a modified transposon end sequence comprises a mosaic end sequence comprising a substitution at C17. In some embodiments, a modified transposon end sequence comprises a mosaic end sequence comprising a substitution at A18. In some embodiments, a modified transposon end sequence comprises a mosaic end sequence comprising a substitution at G19. In some embodiments, the modified transposon end sequence comprises SEQ ID NOs: 5, 7, 9, or 14-22. Data with representative modified transposon end sequence are shown in FIG. 6A (with transposition in solution) and FIG. 12 (with transposition mediated by fBLTs).

In some embodiments, the mosaic end sequence comprises more than one mutation. In some embodiments, the mosaic end sequence comprises no more than 8 mutations as compared to the wild-type sequence (in some embodiment SEQ ID NO: 1).

Additional mutations may also be present in a mosaic end sequence, in addition to the one or more mutations at A16, C17, A18, and/or G19. In some embodiments, the mosaic end sequence comprises one or more mutations as compared to SEQ ID NO: 1 in addition to the one or more mutations at A16, C17, A18, and/or G19. In some embodiments, the mosaic end sequence comprises from one to four substitution mutations as compared to SEQ ID NO: 1 in addition to the one or more mutations at A16, C17, A18, and/or G19.

In some embodiments, the mosaic end sequence has one substitution mutation as compared to SEQ ID NO: 1 in addition to the one or more mutations at A16, C17, A18, and/or G19. In some embodiments, the mosaic end sequence has two substitution mutations as compared to SEQ ID NO: 1 in addition to the one or more mutations at A16, C17, A18, and/or G19. In some embodiments, the mosaic end sequence has three substitution mutations as compared to SEQ ID NO: 1 in addition to the one or more mutations at A16, C17, A18, and/or G19. In some embodiments, the mosaic end sequence has four substitution mutations as compared to SEQ ID NO: 1 in addition to the one or more mutations at A16, C17, A18, and/or G19.

In some embodiments, the substitution at A16 is A16T, A16C, A16G, A16U, A16Inosine, A16Ribose, A16-8-oxoguanine, A16Thymine glycol, A16Modified purine, or A16Modified pyrimidine; the substitution at C17 is C17T, C17A, C17G, C17U, C17Inosine, C17Ribose, C17-8-oxoguanine, C17Thymine glycol, C17Modified purine, or C17Modified pyrimidine; the substitution at A18 is A18G, A18T, A18C, A18U, A18Inosine, A18Ribose, A18-8-oxoguanine, A18Thymine glycol, A18Modified purine, or A18Modified pyrimidine; and/or the substitution at G19 is G19T, G19C, G19A, G19U, G19Inosine, G19Ribose, G19-8-oxoguanine, G19Thymine glycol, G19Modified purine, or G19Modified pyrimidine. In some embodiments, the modified purine is 3mA or 7mG. In some embodiments, the modified pyrimidine is 5-methylcytosine, 5-formylcytosine, or 5-carboxycytosine.

In some embodiments, the mutation comprises a substitution with a uracil; an inosine; a ribose; an 8-oxoguanine; a thymine glycol; a modified purine; and/or a modified pyrimidine. In some embodiments, these mutations allow for methods to cleave the mosaic end sequence after transposition.

In some embodiments, the modified transposon end sequence comprises a mutation at A16, C17, A18, or G19.

In some embodiments, the modified transposon end sequence comprises two mutations chosen from mutations at A16, C17, A18, or G19. In some embodiments, the modified transposon end sequence comprises three mutations chosen from mutations at A16, C17, A18, or G19. In some embodiments, the modified transposon end sequence comprises four mutations at A16, C17, A18, and G19.

In some embodiments, the modified transposon end sequence has from one to four substitution mutations as compared to SEQ ID NO: 1 at A16, C17, A18, and/or G19. In some embodiments, the modified transposon end sequence has one substitution mutation as compared to the wild-type sequence (in some embodiments SEQ ID NO: 1). In some embodiments, the modified transposon end sequence has two substitution mutations as compared to the wild-type sequence (in some embodiments SEQ ID NO: 1). In some embodiments, the modified transposon end sequence has three substitution mutations as compared to the wild-type sequence (in some embodiments SEQ ID NO: 1). In some embodiments, the modified transposon end sequence has four substitution mutations as compared to the wild-type sequence (in some embodiments SEQ ID NO: 1).

### II. Methods of Transposition-Ligation Library Preparation

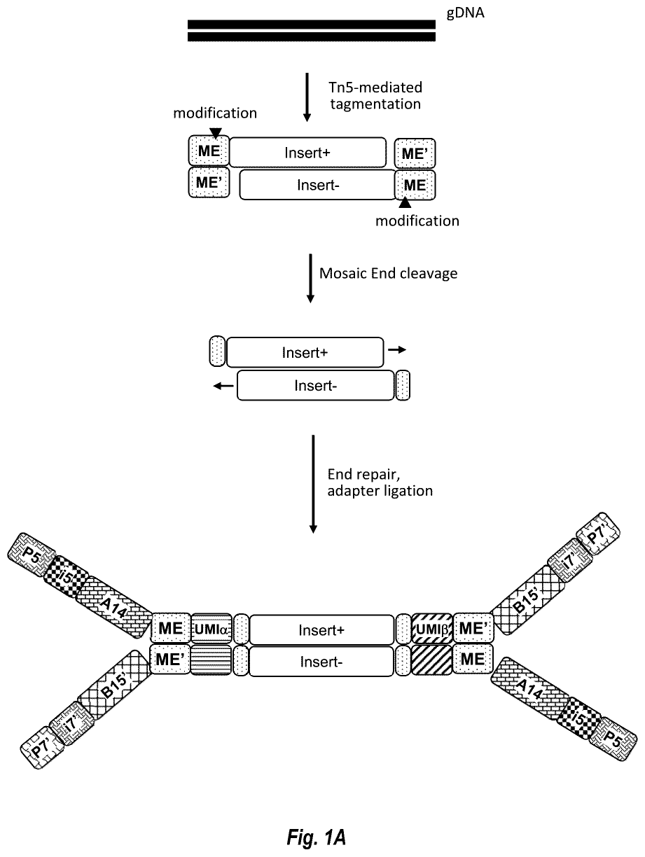

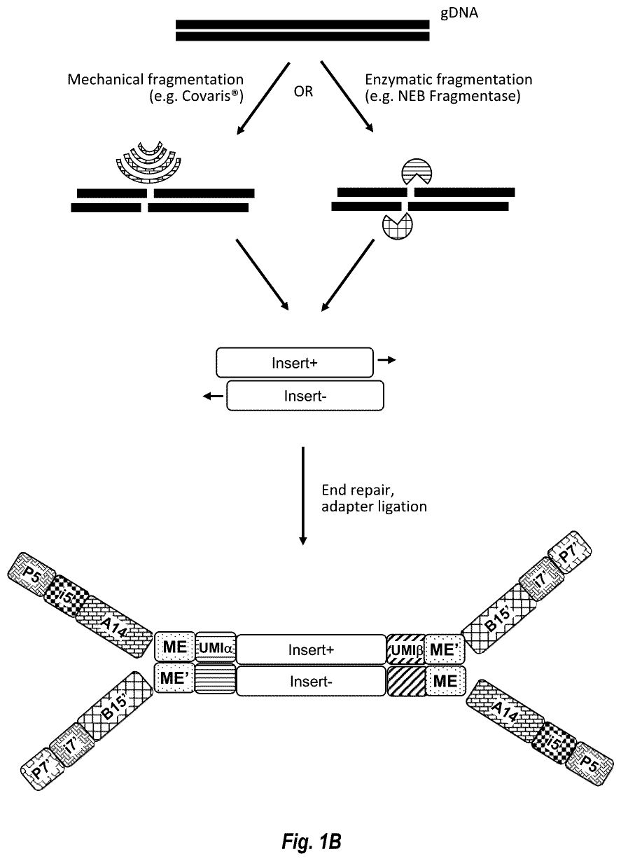

Disclosed herein are methods of library preparation that couple transposition and ligation of adapters. Thus, these library preparation methods may be termed “hybrid transposition-ligation library preparation.” Such methods may use modified Tn5-mosaic end sequences that allow for cleavage of the transferred transposon end after transposition (as shown in FIG. 1A). As used herein, a “hybrid Tn5-ligation approach” refers to a method involving transposition, cleavage of the mosaic end sequence, and ligation of adapters.

In some embodiments, cleavage of the mosaic end sequence allows for its removal from library fragments. While the present methods use ligation after cleavage of the mosaic end sequence, in order to incorporate an adapter for potential downstream sequencing methods, the present method is not limited to embodiments requiring ligation of adapter sequences.

BLTs designed for fragmentation of the mosaic end sequence may be termed “fragmentase BLTs” (fBLTs). While fBLTs do not themselves comprise a fragmentase, fBLTs are designed to prepare fragments that are similar to those prepared with a fragmentase in that the resulting fragments lack all or part of a mosaic end sequence. The fBLTs are designed for cleavage (after transposition) to remove all or part of the mosaic end sequence after fragment generation via transposition.

The present methods can decouple the enzymatic fragmentation and adapter ligation activities of the transposase, such as the Tn5 transposase, through programmed cleavage of the mosaic end sequence from library fragments. As described herein, the transposase, in some embodiments Tn5, can tolerate a number of mutations and nucleobase modifications within the mosaic end substrate. By incorporating modified bases within the transferred strand of the mosaic end, enzymes can enable selective cleavage and removal. This technology eliminates the constraint requiring the 19-bp mosaic end sequence adjacent to the library insert and enables hybrid transposase-ligation library preparation approaches, thus enabling fBLTs to be leveraged in library preparation workflows that have been developed based on ligation chemistries. The present methods thus improve on prior workflows for mechanically shearing or enzymatically fragmenting dsDNA, followed by end repair and adapter ligation (FIG. 1B).

Described herein is a method of preparing double-stranded nucleic acid fragments comprising adapters comprising combining a sample comprising nucleic acid with transposome complexes and preparing fragments; combining the sample with an enzyme or enzyme mixture and cleaving the first transposon end at the uracil, inosine, ribose, 8-oxoguanine, thymine glycol, modified purine, and/or modified pyrimidine within the mosaic end sequence to remove all or part of the first transposon end from the fragments; and ligating an adapter onto the 5′ and/or 3′ ends of the fragments. In some embodiments, the modified purine is 3-methyladenine or 7-methylguanine. In some embodiments, the modified pyrimidine is 5-methylcytosine, 5-formylcytosine, or 5-carboxycytosine. In some embodiments, the enzyme or enzyme mixture is a modification-specific endonuclease or a modification-specific DNA glycosylase. In some embodiments, a modification-specific DNA glycosylase is used together with an endonuclease/lyase, which does not need to be modification-specific. Instead, the endonuclease/lyase recognizes abasic sites.

In some embodiments, the enzyme or enzyme mixture is (1) an endonuclease or (2) a combination of a DNA glycosylase and heat, basic conditions, or an endonuclease/lyase that recognizes abasic sites.

In some embodiments, the method is performed in a single reaction vessel. In other words, methods may be performed without a need to partition reaction products from each other.

### A. Transposome Complexes

A “transposome complex” or “transposome” as used herein, is comprised of at least one transposase (or other enzyme as described herein) and a transposon recognition sequence. The present invention is not limited to a specific transposase.

A “transposome complex” is comprised of at least one transposase enzyme and a transposon recognition sequence. In some such systems, the transposase binds to a transposon recognition sequence to form a functional complex that is capable of catalyzing a transposition reaction. In some aspects, the transposon recognition sequence is a double-stranded transposon end sequence. The transposase, or integrase, binds to a transposase recognition site in a target nucleic acid and inserts the transposon recognition sequence into a target nucleic acid. In some such insertion events, one strand of the transposon recognition sequence (or end sequence) is transferred into the target nucleic acid, resulting also in a cleavage event. Exemplary transposition procedures and systems that can be readily adapted for use with the transposases of the present disclosure are described, for example, in PCT Publ. No. WO10/048605, US Pat. Publ. No. 2012/0301925, US Pat. Publ. No. 2012/13470087, or US Pat. Publ. No. 2013/0143774, each of which is incorporated herein by reference in its entirety.

In some such systems, the transposase binds to a transposon recognition sequence to form a functional complex that is capable of catalyzing a transposition reaction. In some aspects, the transposon recognition sequence is a double-stranded transposon end sequence. The transposase binds to a transposase recognition site in a target nucleic acid and inserts the transposon recognition sequence into a target nucleic acid. In some such insertion events, one strand of the transposon recognition sequence (or end sequence) is transferred into the target nucleic acid, resulting in a cleavage event. Exemplary transposition procedures and systems that can be readily adapted for use with the transposases.

A “transposase” means an enzyme that is capable of forming a functional complex with a transposon end-containing composition (e.g., transposons, transposon ends, transposon end compositions) and catalyzing insertion or transposition of the transposon end-containing composition into a double-stranded target nucleic acid. A transposase as presented herein can also include integrases from retrotransposons and retroviruses.

Exemplary transposases that can be used with certain embodiments provided herein include (or are encoded by): Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, and retrotransposon of yeast. More examples include IS5, Tn10, Tn903, IS911, and engineered versions of transposase family enzymes. The methods described herein could also include combinations of transposases, and not just a single transposase.

In some embodiments, the transposase is a Tn5, Tn7, MuA, or Vibrio harveyi transposase, or an active mutant thereof. In other embodiments, the transposase is a Tn5 transposase or a mutant thereof. In other embodiments, the transposase is a Tn5 transposase or a mutant thereof. In other embodiments, the transposase is a Tn5 transposase or an active mutant thereof. In some embodiments, the Tn5 transposase is a hyperactive Tn5 transposase, or an active mutant thereof. In some aspects, the Tn5 transposase is a Tn5 transposase as described in PCT Publ. No. WO2015/160895, which is incorporated herein by reference. In some aspects, the Tn5 transposase is a hyperactive Tn5 with mutations at positions 54, 56, 372, 212, 214, 251, and 338 relative to wild-type Tn5 transposase. In some aspects, the Tn5 transposase is a hyperactive Tn5 with the following mutations relative to wild-type Tn5 transposase: E54K, M56A, L372P, K212R, P214R, G251R, and A338V. In some embodiments, the Tn5 transposase is a fusion protein. In some embodiments, the Tn5 transposase fusion protein comprises a fused elongation factor Ts (Tsf) tag. In some embodiments, the Tn5 transposase is a hyperactive Tn5 transposase comprising mutations at amino acids 54, 56, and 372 relative to the wild type sequence. In some embodiments, the hyperactive Tn5 transposase is a fusion protein, optionally wherein the fused protein is elongation factor Ts (Tsf). In some embodiments, the recognition site is a Tn5-type transposase recognition site (Goryshin and Reznikoff, J. Biol. Chem., 273:7367, 1998). In one embodiment, a transposase recognition site that forms a complex with a hyperactive Tn5 transposase is used (e.g., EZ-Tn5TM Transposase, Epicentre Biotechnologies, Madison, Wis.). In some embodiments, the Tn5 transposase is a wild-type Tn5 transposase.

As used throughout, the term transposase refers to an enzyme that is capable of forming a functional complex with a transposon-containing composition (e.g., transposons, transposon compositions) and catalyzing insertion or transposition of the transposon-containing composition into the double-stranded target nucleic acid with which it is incubated in an in vitro transposition reaction. A transposase of the provided methods also includes integrases from retrotransposons and retroviruses. Exemplary transposases that can be used in the provided methods include wild-type or mutant forms of Tn5 transposase and MuA transposase.

A “transposition reaction” is a reaction wherein one or more transposons are inserted into target nucleic acids at random sites or almost random sites. Essential components in a transposition reaction are a transposase and DNA oligonucleotides that exhibit the nucleotide sequences of a transposon, including the transferred transposon sequence and its complement (i.e., the non-transferred transposon end sequence) as well as other components needed to form a functional transposition or transposome complex. The method of this disclosure is exemplified by employing a transposition complex formed by a hyperactive Tn5 transposase and a Tn5-type transposon end or by a MuA or HYPERMu transposase and a Mu transposon end comprising R1 and R2 end sequences (See e.g., Goryshin, I. and Reznikoff, W. S., J. Biol. Chem., 273: 7367, 1998; and Mizuuchi, Cell, 35: 785, 1983; Savilahti, H, et al., EMBO J., 14: 4893, 1995; which are incorporated by reference herein in their entireties). However, any transposition system that is capable of inserting a transposon end in a random or in an almost random manner with sufficient efficiency to tag target nucleic acids for its intended purpose can be used in the provided methods. Other examples of known transposition systems that could be used in the provided methods include but are not limited to Staphylococcus aureus Tn552, Ty1, Transposon Tn7, Tn/O and IS 10, Mariner transposase, Tel, P Element, Tn3, bacterial insertion sequences, retroviruses, and retrotransposon of yeast (See, e.g., Colegio O R et al, J. Bacteriol., 183: 2384-8, 2001; Kirby C et al, Mol. Microbiol., 43: 173-86, 2002; Devine S E, and Boeke J D., Nucleic Acids Res., 22: 3765- 72, 1994; International Patent Application No. WO 95/23875; Craig, N L, Science. 271 : 1512, 1996; Craig, N L, Review in: Curr Top Microbiol Immunol., 204: 27-48, 1996; Kleckner N, et al., Curr Top Microbiol Immunol., 204: 49-82, 1996; Lampe D J, et al., EMBO J., 15: 5470-9, 1996; Plasterk R H, Curr Top Microbiol Immunol, 204: 125-43, 1996; Gloor, G B, Methods Mol. Biol, 260: 97-1 14, 2004; Ichikawa H, and Ohtsubo E., J Biol. Chem. 265: 18829-32, 1990; Ohtsubo, F and Sekine, Y, Curr. Top. Microbiol. Immunol. 204: 1-26, 1996; Brown P O, et al, Proc Natl Acad Sci USA, 86: 2525-9, 1989; Boeke J D and Corces V G, Annu Rev Microbiol. 43: 403-34, 1989; which are all incorporated herein by reference in their entireties).

The method for inserting a transposon into a target sequence can be carried out in vitro using any suitable transposon system for which a suitable in vitro transposition system is available or can be developed based on knowledge in the art. In general, a suitable in vitro transposition system for use in the methods of the present disclosure requires, at a minimum, a transposase enzyme of sufficient purity, sufficient concentration, and sufficient in vitro transposition activity and a transposon with which the transposase forms a functional complex with the respective transposase that is capable of catalyzing the transposition reaction. Suitable transposase transposon end sequences that can be used include but are not limited to wild-type, derivative or mutant transposon end sequences that form a complex with a transposase chosen from among a wild-type, derivative, or mutant form of the transposase.

In some embodiments, the transposase comprises a Tn5 transposase. In some embodiments, the Tn5 transposase is hyperactive Tn5 transposase.

In some embodiments, the transposome complex comprises a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. In some embodiments, the transposome complexes in each population are homodimers, wherein the first population has a first adapter sequence in each monomer and the second population has a different adapter sequence in each monomer.

The term “transposon end” refers to a double-stranded nucleic acid DNA that exhibits only the nucleotide sequences (the “transposon end sequences”) that are necessary to form the complex with the transposase or integrase enzyme that is functional in an in vitro transposition reaction. In some embodiments, a transposon end is capable of forming a functional complex with the transposase in a transposition reaction. As non-limiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US 2010/0120098, the content of which is incorporated herein by reference in its entirety. Transposon ends can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands. Although the term “DNA” is used throughout the present disclosure in connection with the composition of transposon ends, it should be understood that any suitable nucleic acid or nucleic acid analogue can be utilized in a transposon end.

The term “transferred strand” refers to the portion of a pair of transposons that is transferred to a fragment of nucleic acid from a sample during a transposition reaction. Similarly, the term “non-transferred strand” refers to the portion of a pair of transposons that is not transferred to a fragment of nucleic acid from a sample during a transposition reaction. Within a pair of transposons, the transferred strand and non-transferred strands may be all or partially complementary. The 3′-end of a transferred strand is joined or transferred to target DNA in an in vitro transposition reaction. The non-transferred strand, which exhibits a transposon end sequence that is all or partially complementary to the transferred transposon end sequence, is not joined or transferred to the target DNA in an in vitro transposition reaction.

In some embodiments, the transferred strand and non-transferred strand are covalently joined. For example, in some embodiments, the transferred and non-transferred strand sequences are provided on a single oligonucleotide, e.g., in a hairpin configuration. As such, although the free end of the non-transferred strand is not joined to the target DNA directly by the transposition reaction, the non-transferred strand becomes attached to the DNA fragment indirectly, because the non-transferred strand is linked to the transferred strand by the loop of the hairpin structure. Additional examples of transposome structure and methods of preparing and using transposomes can be found in the disclosure of US 2010/0120098, the content of which is incorporated herein by reference in its entirety.

As used herein, a “transposome complex” and a “transposome” are equivalent.

In some embodiments, the first transposon comprises the transferred strand in the transposition reaction. In some embodiments, the second transposon comprises the non-transferred strand in the transposition reaction.

In some embodiments, the transposomes comprise a modified transposon end with mutations in the mosaic end sequence.

In some embodiments, a transposome complex comprises a transposase; a first transposon comprising a modified transposon end sequence comprising a uracil, an inosine, a ribose, an 8-oxoguanine, a thymine glycol, a modified purine, and/or a modified pyrimidine; and a second transposon comprising a second transposon end sequence complementary to at least a portion of the first transposon end sequence.

In some embodiments, the first transposon comprises a ribose and the transposome complex is in solution. In some embodiments, the first transposon comprises a uracil, an inosine, an 8-oxoguanine, a thymine glycol, a modified purine, and/or a modified pyrimidine and the transposome complex is immobilized on a solid support.

In some embodiments, the first transposon comprises a modified transposon end sequence. In some embodiments, the transposase is Tn5. In some embodiments, the first transposon is the transferred strand. In some embodiments, the second transposon is the non-transferred strand.

In some embodiments, a uracil in the first transposon is base paired with an A in the second transposon. In some embodiments, an inosine in the first transposon is base paired with a C in the second transposon. In some embodiments, a ribose in the first transposon is base paired with an A, C, T, or G in the second transposon. In some embodiments, a thymine glycol in the first transposon is base paired with an A in the second transposon. In some embodiments, the modified purine is a 3-methyladenine in the first transposon that is base paired with an T in the second transposon. In some embodiments, the modified purine is a 7-methylguanine in the first transposon that is base paired with a C in the second transposon. In some embodiments, the modified pyrimidine is a 5-methylcytosine, 5-formylcytosine, or 5-carboxycytosine in the first transposon that is base paired with a G in the second transposon.

In some embodiments, the second transposon comprises the sequence complementary to SEQ ID NO: 1. In some embodiments, the second transposon comprises a second transposon end sequence complementary to SEQ ID NO: 1. In some embodiments, the second transposon comprises SEQ ID NO: 4.

In some situations, the second transposon end may be fully complementary to the first transposon end, while in other situations it may be partially complementary. While not being bound by theory, a transposase may have greater activity when a pair of transposon ends (i.e., the first transposon and second transposon) comprise fewer mutations. For example, a transposome complex comprising a second transposon comprising a sequence that is complementary to SEQ ID NO: 1 (i.e., the complement of the wild-type mosaic end sequence) may mediate greater activity than a transposome complex comprising a second transposon end that is complementary to a first transposon end comprising a modified transposon end sequence as described herein. In other situations, the second transposon may be fully complementary to the first transposon to promote tighter annealing of the transposon pair.

In some embodiments, the first transposon comprises a modified transposon end sequence comprising an A16U, A16-8-oxoguanine, or A16Inosine substitution as compared to SEQ ID NO: 1 and the second transposon comprises a second transposon end sequence complementary to SEQ ID NO: 1 or a second transposon end fully complementary to the first transposon end.

In some embodiments, the first transposon comprises a modified transposon end sequence comprising an C17-8-oxoguanine or C17Inosine substitution as compared to SEQ ID NO: 1 and the second transposon comprises a second transposon end sequence complementary to SEQ ID NO: 1 or a second transposon end fully complementary to the first transposon end.

In some embodiments, the first transposon comprises a modified transposon end sequence comprising an A18-8-oxoguanine or A18Inosine substitution as compared to SEQ ID NO: 1 and the second transposon comprises a second transposon end sequence complementary to SEQ ID NO: 1 or a second transposon end fully complementary to the first transposon end.

In some embodiments, the first transposon comprises a modified transposon end sequence comprising an G19-8-oxoguanine or G19Inosine substitution as compared to SEQ ID NO: 1 and the second transposon comprises a second transposon end sequence complementary to SEQ ID NO: 1 or a second transposon end fully complementary to the first transposon end.

In some embodiments, a transposome complex comprises a pair of transposons comprising a first transposon and a second transposon, wherein the first transposon comprises a modified transposon end sequence as described herein and wherein the second transposon comprises a transposon end sequence comprising a mosaic end sequence complementary to a wild-type mosaic end sequence. A pair of transposons may comprise any modified transposon end sequence described herein.

In some embodiments, the first transposon comprises a modified transposon end sequence comprising an A16U, A16-8-oxoguanine, or A16Inosine substitution as compared to SEQ ID NO: 1 and the second transposon comprises a second transposon end sequence complementary to SEQ ID NO: 1 or a second transposon end fully complementary to the first transposon end.

In some embodiments, the first transposon comprises a modified transposon end sequence comprising an C17-8-oxoguanine or C17Inosine substitution as compared to SEQ ID NO: 1 and the second transposon comprises a second transposon end sequence complementary to SEQ ID NO: 1 or a second transposon end fully complementary to the first transposon end.

In some embodiments, the first transposon comprises a modified transposon end sequence comprising an A18-8-oxoguanine or A18Inosine substitution as compared to SEQ ID NO: 1 and the second transposon comprises a second transposon end sequence complementary to SEQ ID NO: 1 or a second transposon end fully complementary to the first transposon end.

In some embodiments, the first transposon comprises a modified transposon end sequence comprising an G19-8-oxoguanine or G19Inosine substitution as compared to SEQ ID NO: 1 and the second transposon comprises a second transposon end sequence complementary to SEQ ID NO: 1 or a second transposon end fully complementary to the first transposon end.

In some embodiments, there is a mismatch between the first transposon and second transposon at the position wherein the first transposon comprises a mutation as compared to a wild-type mosaic end sequence. In other words, the first transposon and second transposon do not need to be fully complementary (i.e., a U in a first transposon does not need to base with an A in the second transposon).

### 1. Transposome Complexes in Solution

In some embodiments, a transposome complex is in solution, which may be referred to as a solution-phase transposome complex or soluble transposome complex. In some embodiments, double-stranded nucleic acid (such as DNA) bound to solution-phase transposomes complexes undergoes tagmentation to yield nucleic acid fragments that are free in solution. Such a process, referred to herein as “tagmentation,” often involves the modification of DNA by a transposome complex comprising transposase enzyme complexed with adaptors comprising transposon end sequence. In some embodiments, the solution is a tagmentation buffer.

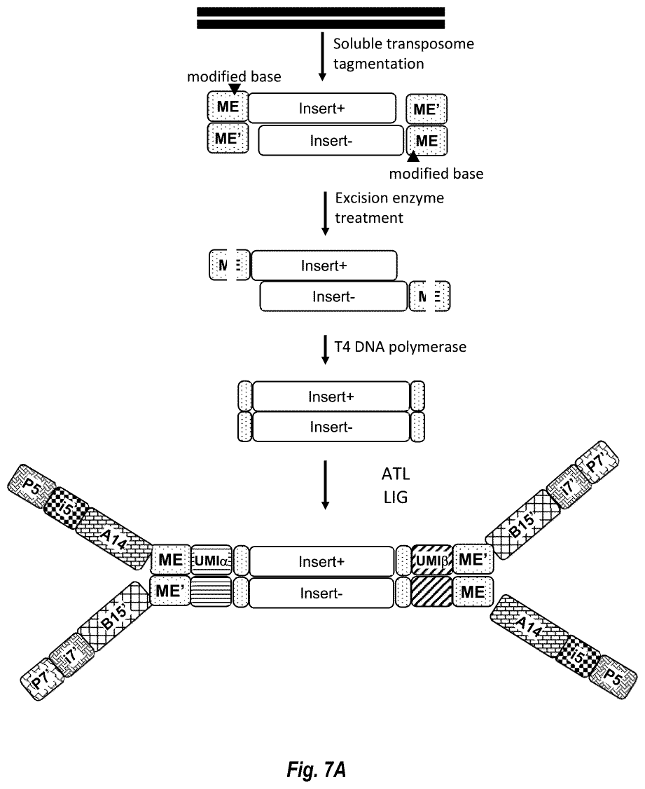

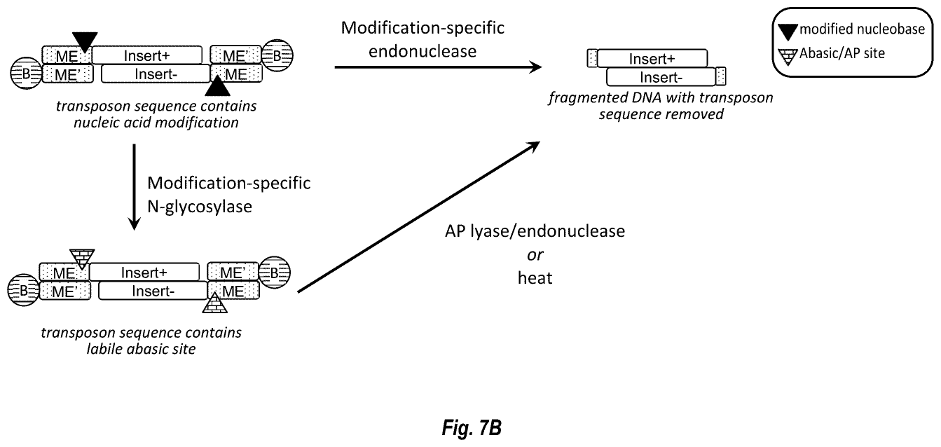

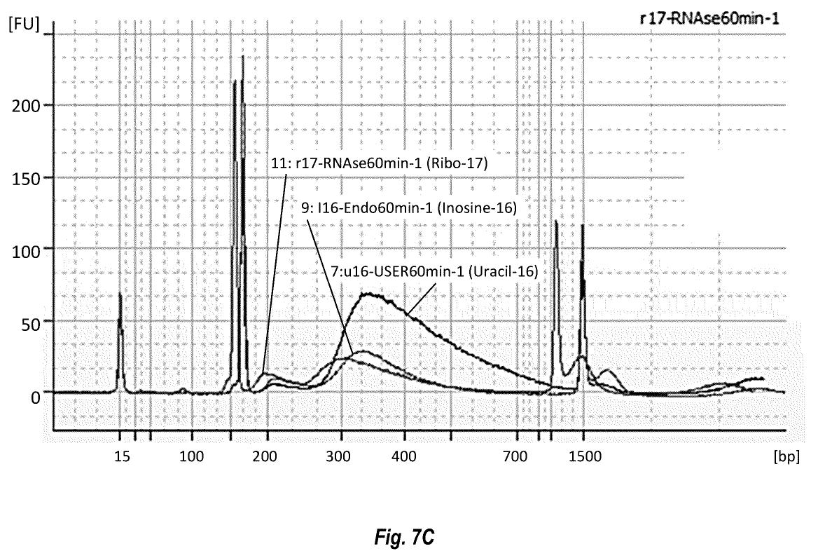

Protocols available for soluble tagmentation are well-known in the art, such as those described for the Illumina DNA Nextera® XT DNA Library Preparation Kit (see Nextera XT Reference Guide, Document 770-2012-011). Representative data with soluble tagmentation are shown in FIGS. 7C-9.

In some embodiments, certain modified transposon ends may perform better when the transposition reaction is performed in solution. For example, modified transposon ends comprising ribose may perform better when comprised in transposome complexes in solution as compared to when transposome complexes are immobilized on a solid support.

In some embodiments, the modified transposon end comprised in a solution-phase transposome comprises a uracil, an inosine, an 8-oxoguanine, a thymine glycol, a modified purine, and/or a modified pyrimidine. In some embodiments, the modified transposon end comprised in a solution-phase transposome complex comprises ribose.

In another example, modified transposon ends comprising modifications at position 16 of SEQ ID NO: 1 may perform better when comprised in transposome complexes in solution as compared to when comprised in transposome complexes immobilized on a solid support. This difference may be due to a number of factors, such the affinity of different modified transposon ends for transposases and the procedure used for the preparation of the bead-linked transposome.

### 2. Immobilized Transposome Complexes and Bead-Linked Transposomes

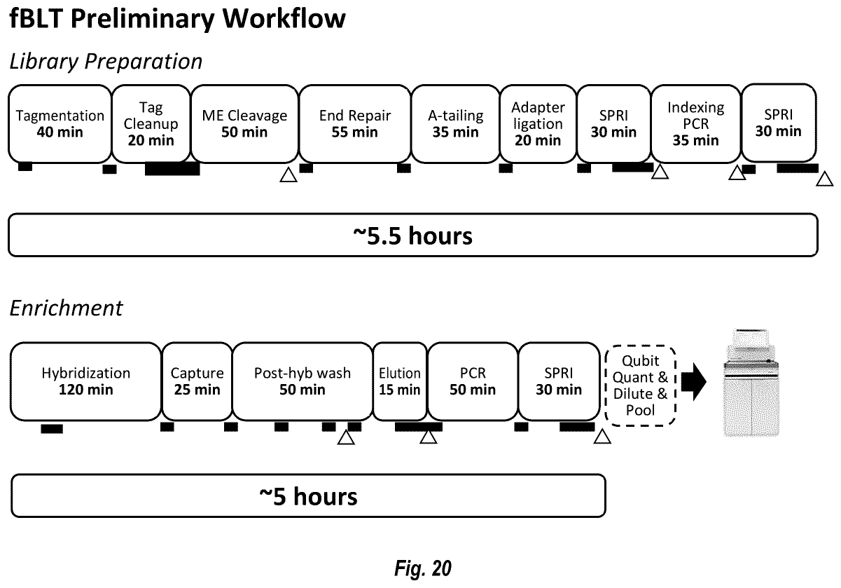

In some embodiments, a transposome complex is immobilized to a solid support. In some embodiments, the solid support is comprised within a tagmentation buffer. In some embodiments, double-stranded nucleic acid (such as DNA) bound to immobilized transposomes complexes undergoes tagmentation to yield immobilized nucleic acid fragments. Such bead-immobilized transposome complexes that can be used for fragmentation may be termed “fBLTs.” A representative protocol for performing library preparation with fBLTs is shown in FIG. 20.

In some embodiments, the first transposon comprises a uracil, an inosine, an 8-oxoguanine, a thymine glycol, a modified purine, and/or a modified pyrimidine and the transposome complex is immobilized on a solid support.

In some embodiments, the density of transposomes immobilized on the solid surface is selected to modulate fragment size and library yield of the immobilized fragments. In some embodiments, the transposome complexes are present on the solid support at a density of at least 10³, 10⁴, 10^{5,} or 10⁶ complexes per mm².

In some embodiments, the lengths of the double-stranded nucleic acid fragments in the immobilized library are adjusted by increasing or decreasing the density of transposome complexes on the solid support.

A number of different types of immobilized transposomes can be used in these methods, as described in U.S. Pat. No. 9,683,230, which is incorporated herein in its entirety.

In the methods and compositions presented herein, transposome complexes are immobilized to the solid support. In some embodiments, the transposome complexes and/or capture oligonucleotides are immobilized to the support via one or more polynucleotides, such as a polynucleotide comprising a transposon end sequence. In some embodiments, the transposome complex may be immobilized via a linker molecule coupling the transposase enzyme to the solid support. In some embodiments, both the transposase enzyme and the polynucleotide are immobilized to the solid support. When referring to immobilization of molecules (e.g. nucleic acids) to a solid support, the terms “immobilized” and “attached” are used interchangeably herein and both terms are intended to encompass direct or indirect, and covalent or non-covalent attachment, unless indicated otherwise, either explicitly or by context. In some embodiments, covalent attachment may be used, but generally all that is required is that the molecules (e.g. nucleic acids) remain immobilized or attached to the support under the conditions in which it is intended to use the support, for example in applications requiring nucleic acid amplification and/or sequencing.

Certain embodiments may make use of solid supports comprised of an inert substrate or matrix (e.g. glass slides, polymer beads etc.) which has been functionalized, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (e.g. polynucleotides) may be directly covalently attached to the intermediate material (e.g. the hydrogel) but the intermediate material may itself be non-covalently attached to the substrate or matrix (e.g. the glass substrate). The term “covalent attachment to a solid support” is to be interpreted accordingly as encompassing this type of arrangement.

The terms “solid surface,” “solid support” and other grammatical equivalents herein refer to any material that is appropriate for or can be modified to be appropriate for the attachment of the transposome complexes. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. Particularly useful solid supports and solid surfaces for some embodiments are located within a flow cell apparatus. Exemplary flow cells are set forth in further detail below.

In some embodiments, the solid support comprises a patterned surface suitable for immobilization of transposome complexes in an ordered pattern. A “patterned surface” refers to an arrangement of different regions in or on an exposed layer of a solid support. For example, one or more of the regions can be features where one or more transposome complexes are present. The features can be separated by interstitial regions where transposome complexes are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the transposome complexes are randomly distributed upon the solid support. In some embodiments, the transposome complexes are distributed on a patterned surface. Exemplary patterned surfaces that can be used in the methods and compositions set forth herein are described in U.S. application Ser. No. 13/661,524 or US Pat. App. Publ. No. 2012/0316086 A1, each of which is incorporated herein by reference.

In some embodiments, the solid support comprises an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The composition and geometry of the solid support can vary with its use. In some embodiments, the solid support is a planar structure such as a slide, chip, microchip and/or array. As such, the surface of a substrate can be in the form of a planar layer. In some embodiments, the solid support comprises one or more surfaces of a flow cell. The term “flow cell” as used herein refers to a chamber comprising a solid surface across which one or more fluid reagents can be flowed. Examples of flow cells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In some embodiments, the solid support or its surface is non-planar, such as the inner or outer surface of a tube or vessel. In some embodiments, the solid support comprises microspheres or beads. By “microspheres” or “beads” or “particles” or grammatical equivalents herein is meant small discrete particles. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and teflon, as well as any other materials outlined herein for solid supports may all be used. “Microsphere Selection Guide” from Bangs Laboratories, Fishers Ind. is a helpful guide. In certain embodiments, the microspheres are magnetic microspheres or beads.

The beads need not be spherical; irregular particles may be used. Alternatively or additionally, the beads may be porous. The bead sizes range from nanometers, i.e. 100 nm, to millimeters, i.e. 1 mm, with beads from 0.2 micron to 200 microns, or from 0.5 to 5 microns, although in some embodiments smaller or larger beads may be used.

In some embodiments, on-bead tagmentation allows for a more uniform tagmentation reaction compared to in-solution tagmentation reactions.

The density of these surface-bound transposomes can be modulated by varying the density of the first polynucleotide or by the amount of transposase added to the solid support. For example, in some embodiments, the transposome complexes are present on the solid support at a density of at least 103, 104, 105, or 106 complexes per mm².

Attachment of a nucleic acid to a support, whether rigid or semi-rigid, can occur via covalent or non-covalent linkage(s). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference. In some embodiments, a nucleic acid or other reaction component can be attached to a gel or other semisolid support that is in turn attached or adhered to a solid-phase support. In such embodiments, the nucleic acid or other reaction component will be understood to be solid phase.

In some embodiments, the solid support comprises microparticles, beads, a planar support, a patterned surface, or wells. In some embodiments, the planar support is an inner or outer surface of a tube.

In some embodiments, a solid support has a library of tagged DNA fragments immobilized thereon prepared.

In some embodiments, solid support comprises capture oligonucleotides and a first polynucleotide immobilized thereon, wherein the first polynucleotide comprises a 3′ portion comprising a transposon end sequence and a first tag.

In some embodiments, the solid support further comprises a transposase bound to the first polynucleotide to form a transposome complex.

In some embodiments, a solid support comprises capture oligonucleotides and a second polynucleotide immobilized thereon, wherein the second polynucleotide comprises a 3′ portion comprising a transposon end sequence and a second tag.

In some embodiments, the solid support further comprises a transposase bound to the second polynucleotide to form a transposome complex.

In some embodiments, a kit comprises a solid support as described herein. In some embodiments, a kit further comprises a transposase. In some embodiments, a kit further comprises a reverse transcriptase polymerase. In some embodiments, a kit further comprises a second solid support for immobilizing DNA.

A wide variety of different means of immobilizing transposome complexes have been described, such as those described in WO 2018/156519, which is incorporated herein in its entirety. In some embodiments, the first transposon comprised in the transposome complex comprises an affinity element. In some embodiments, the affinity element is attached to the 5′ end of the first transposon. In some embodiments, the first transposon comprises a linker. In some embodiments, the linker has a first end attached to the 5′ end of the first transposon and a second end attached to an affinity element.

In some embodiments, the transposome complex further comprises a second transposon complementary to at least a portion of the first transposon end sequence. In some embodiments, the second transposon comprises an affinity element. In some embodiments, the affinity element is attached to the 3′ end of the second transposon. In some embodiments, the second transposon comprises SEQ ID NO: 13. In some embodiments, the second transposon comprises a linker. In some embodiments, the linker has a first end attached to the 3′ end of the second transposon and a second end attached to an affinity element.

In some embodiments, the affinity element comprises biotin, avidin, streptavidin, an antibody, or an oligonucleotide. In some embodiments, the affinity element is biotin. In some embodiments, the affinity element comprises oligonucleotide that can bind to a capture oligonucleotide comprised on the surface of a solid support. In some embodiments, the affinity element comprises an antibody that can bind to a ligand comprised on the surface of a solid support.

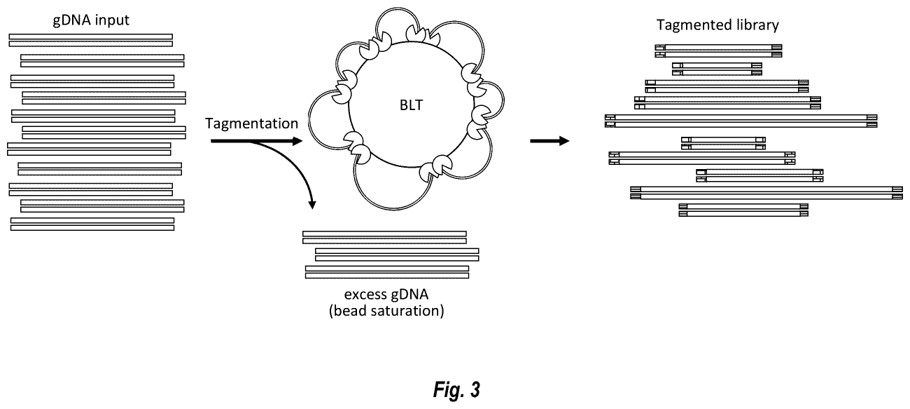

As used herein, “bead-linked transposomes” of “BLTs” refer to transposomes immobilized on beads. Bead-linked transposomes (BLTs) are a key technology in certain NGS library preparation methods, such as Illumina's library preparation products. Bead-linked transposomes leverage the unique advantages of enzymatic Tn5-mediated tagmentation, with additional advantages of providing library normalization and obviating the need for input DNA quantification (FIG. 3 and Stephen Bruinsma et al., BMC Genomics 19(1):1-16 (2018)). A disadvantage of solution-based tagmentation protocols is that control of the ratio between genomic DNA substrate and Tn5 enzyme directly effects library fragment size, resulting in a source of variability in performance. By conjugating a predetermined amount of transposome to a solid support, BLTs enable greater control of library fragment size. Furthermore, the known quantity of transposome bound to the beads provides an upper limit in the amount of DNA substrate that can be converted into library, leading to library normalization.

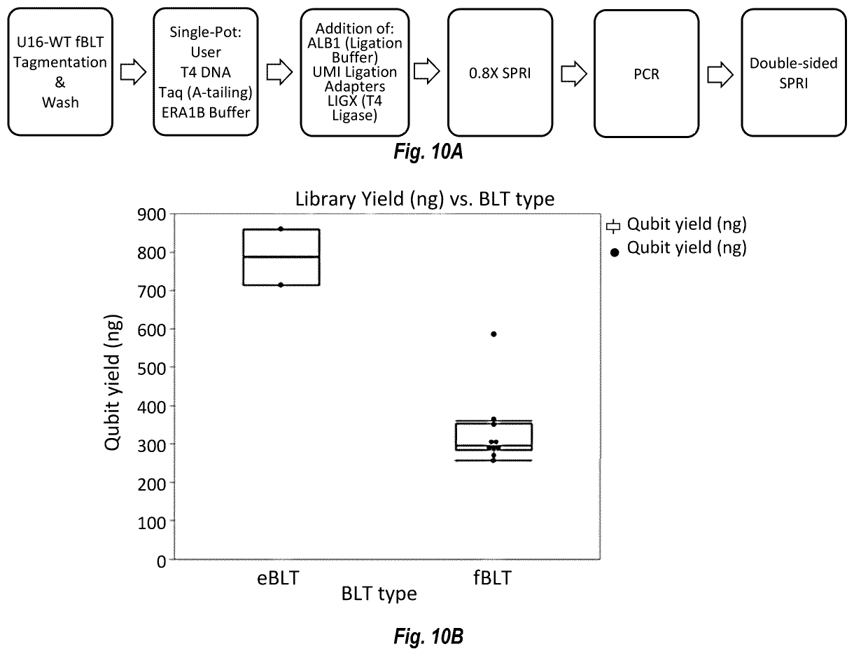

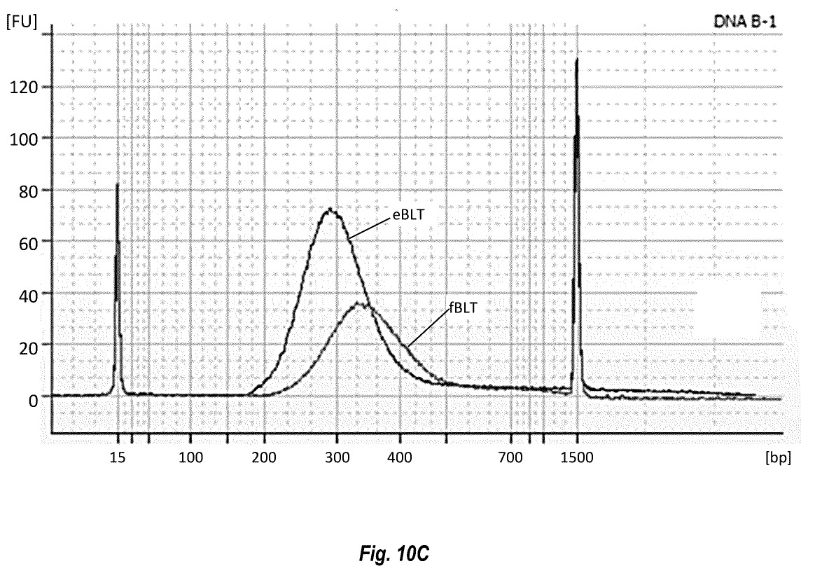

In some embodiments, a solid support comprises transposome complexes described herein immobilized thereon. In some embodiments, the solid support comprises beads (i.e., a fBLT). Representative data generated with fBLTs are shown in FIGS. 10A-18B.

### B. Transposition Reactions for Fragmenting

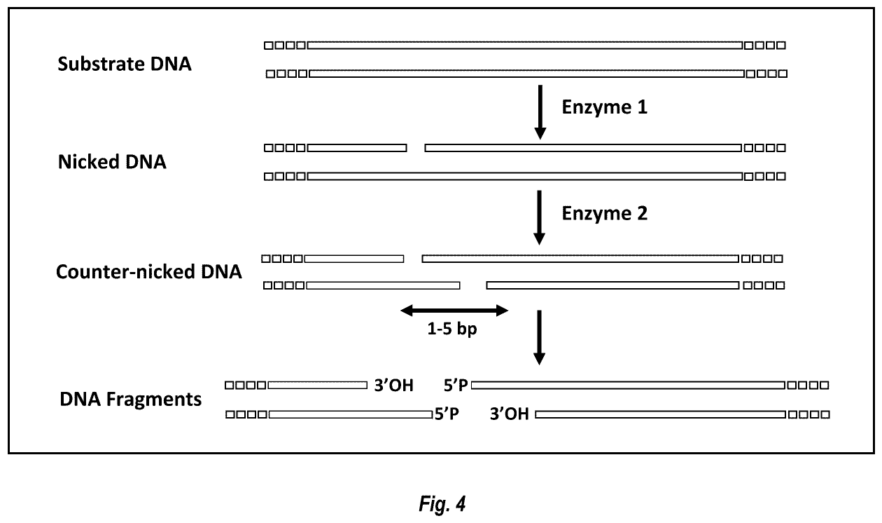

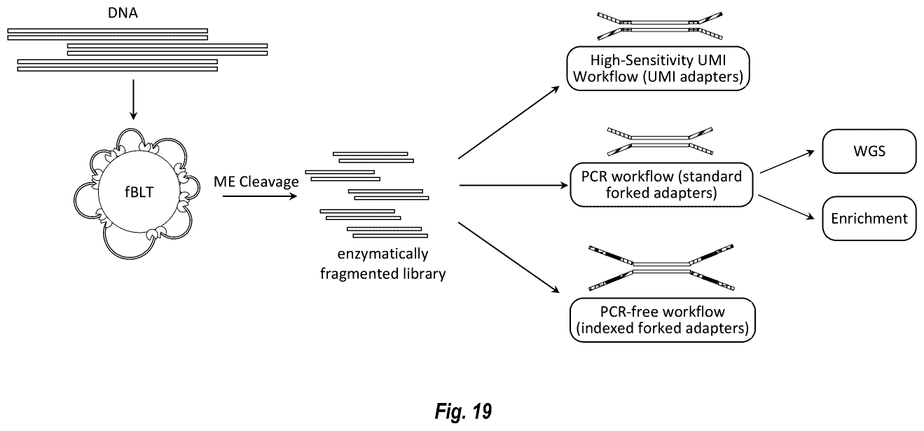

Transposition is an enzyme-mediated process by which DNA sequences are inserted, deleted, and duplicated within genomes. This process has been adapted for broad uses in fragmented double-stranded nucleic acids (such as double-stranded DNA and DNA:RNA duplexes). Transposition can generate DNA fragments without using the standard fragmentase protocols outlined in FIG. 4. In some embodiments, methods of preparing library fragments using modified transposon end sequences are performed with transposomes immobilized on a solid support (such as fBLTs, as shown in FIG. 19). A method of library preparation with fBLTs may take approximately 5.5 hours (as shown in FIG. 20), which is similar to other ligation-based library preparation methods.

In some embodiments, generation of fragments by the present methods with modified transposon ends (such as with fBLTs) avoids DNA damage associated with oxidation during sonication. Such oxidative DNA damage generated from sonication is well-known in the art (see, for example, Costello Nucleic Acids Research 41(6):e67 (2013)). For example, use of fBLTs led to an approximately 50-fold reduction in false-positive G>T transversions, as these transversions are likely driven by oxidative damage to guanine during sonication.

While this transposition reaction will be described with Tn5, other transposases (as described below) can mediate similar reactions.

The well-studied E. coli Tn5 transposon mobilizes by a “cut-and-paste” transposition mechanism. First, the Tn5 transposase Tnp (hereafter, referred to as Tn5) recognizes conserved substrate sequences on either side of transposon DNA, which is then excised, or “cut” from the genome. Tn5 then inserts, or “pastes” this transposon DNA into a target DNA.

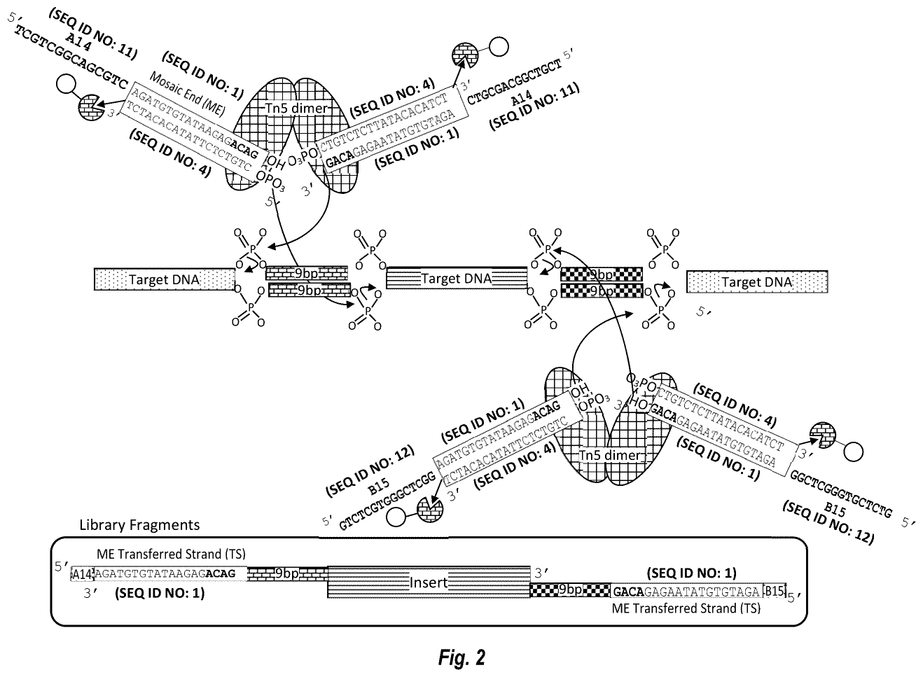

Tn5 has been leveraged in many library preparation reagents (such as those of Illumina) for its ability to “tagment,” that is, simultaneously “tag” and “fragment” genomic DNA, thus greatly decreasing the time and complexity involved in conventional sonication/ligation-based library preparation protocols. In order to support its use with library preparation, Tn5 is pre-loaded with transposons consisting of the conserved substrate sequence, called a “mosaic end” or “end sequence” appended to adapter sequences (e.g., Illumina's A14 and B15 adapter sequences). Then, this transposome complex, comprising the Tn5 transposase and the adapter-bearing transposon sequence, is mixed with a genomic DNA sample. Resulting library preparation transposons bear only short adapter sequences, thus simultaneously leading to fragmentation of the genomic DNA and tagging with the short adapter sequences (FIG. 2).

In some embodiments, transposition with the modified transposon ends described herein gives comparable results as transposition with a wild-type (i.e., transposon end not comprising a mutation described herein). In some embodiments, preparing fragments with a transposome complex described herein leads to preparation of at least 50%, at least 60%, at least 70%, at least 80%, or at least 90% the number of fragments, as compared with preparing fragments with a transposome complex that comprises a first transposon comprising a transposon end sequence comprising a wildtype mosaic end sequence comprising SEQ ID No: 1.

In some embodiments, transposition reactions are performed with transposome complexes comprising a modified transposon end at the 3′ end of the transferred strand.

### 1. Library Fragments Generated by Single Tagmentation Events

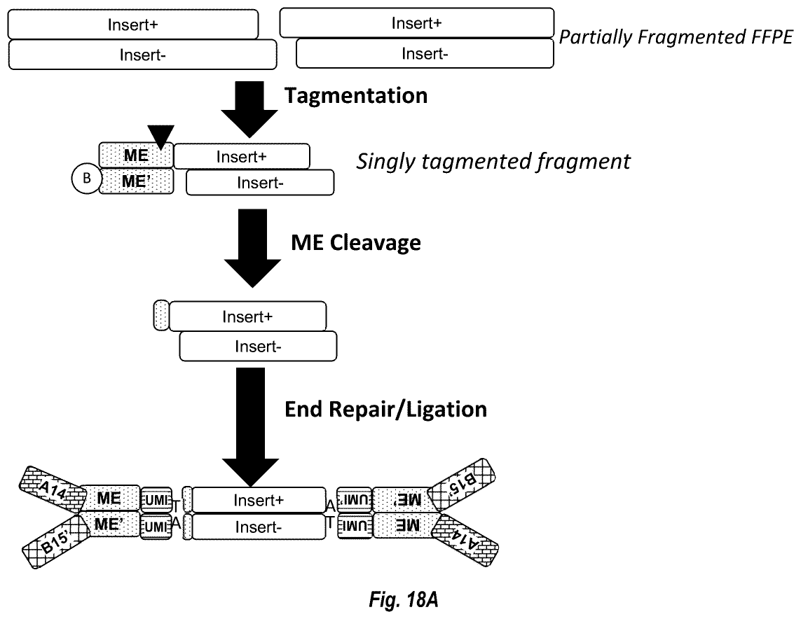

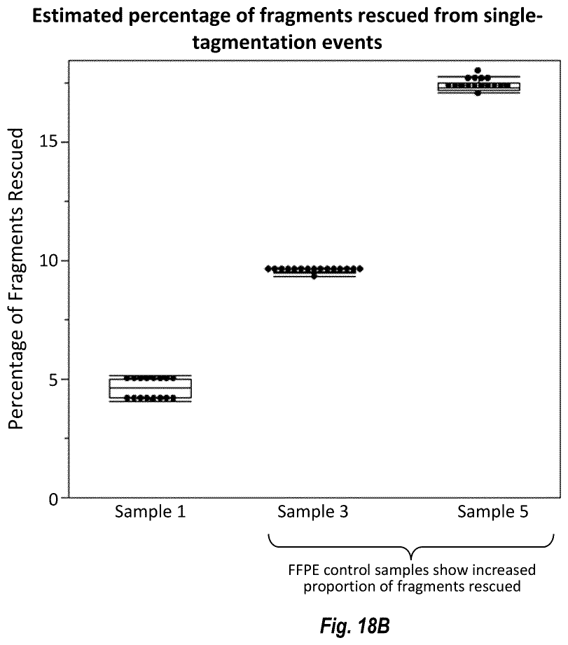

Normally, tagmentation methods for library preparation need to tag both ends of fragments to incorporate adapter sequences used for sequencing methods. However, the present fragmentation methods (such as with fBLTs) allow the possibility to prepare fragments by a single tagmentation event, wherein a mosaic end sequence is added only one end of a fragment. After tagmentation/cleavage with a single tagmentation event, both ends of the fragment can undergo end-repair followed by ligation of adapters as shown in FIG. 18A. Since single tagmentation events normally yield unsequenceable fragments with standard methods (since an adapter sequence would only be incorporated at one end), the ability to sequence fragments after a single tagmentation event may be termed “rescue” of single tagmentation events (as shown in the data in FIG. 18B).

In some embodiments, a transposition reaction for fragmenting improves library preparation from samples comprising partially fragmented nucleic acid. In some embodiments, transposition reactions for fragmenting can be used to fragment one end of DNA molecule followed by end repair and ligation of adapters at both the fragmented and unfragmented end of the molecule. In such a workflow, as shown in FIG. 18A, cleavage of the ME sequence is only performed at one end of a fragment, but the other end of the fragment can also be end repaired followed by adapter ligation. In this way, adapters are ligated to both ends of fragments.

In some embodiments, an fBLT workflow allows for rescue of library fragments prepared with a single tagmentation event. Rescue of library fragments prepared with a single tagmentation event may especially improve results for samples that comprise partially fragmented DNA. This is because fragments prepared by two tagmentation events from partially fragmented DNA may be shorter than the preferred length for sequencing, resulting in loss of successful sequencing. This effect may be offset in part by the ability to rescue single tagmentation events using the methods described herein.

In some embodiments, the sample comprises partially fragmented DNA. In some embodiments, the sample comprising partially fragmented DNA is formalin fixed paraffin embedded (FFPE) tissue or cell-free DNA. In some embodiments, the library comprises fragments prepared by a single tagmentation event.

### 2. Normalization With fBLTs

The presently described fBLTs may be used for library normalization. “Normalization” or “library normalization,” as used herein, refers to the process of diluting libraries of variable 441 concentration to the same or a similar concentration before volumetric pooling.

In some embodiments, normalization helps to ensure an even read distribution for all samples during sequencing. In other words, normalizing libraries can help to ensure even representation in the final sequencing data. In some embodiments, using fBLTs for library normalization avoids downstream steps of a manual normalization protocol.

The requirements for manually normalizing library concentrations are well-known in the art (see, for example Best Practices for Manually Normalizing Library Concentrations, Illumina, Apr. 22, 2021). In some embodiments, a method of normalizing with fBLTs does not require calculation of the library concentration. In this way, a user may avoid time-consuming and cumbersome calculations and dilutions during normalization.

Some bead-linked transposome (BLT) methodologies allow for bead-based normalization, such as Illumina® DNA Prep, (M) Tagmentation (formerly known as Nextera DNA Flex). In some embodiments, fBLTs similarly allow for bead-based normalization. The ability to normalize with a bead-based method avoids time and potential sample loss from performing a separate normalization protocol after library preparation.

### C. Tunable Library Fragment Sizing Using fBLTs

In some embodiments, fBLTs (in lieu of solution-phase transposomes) generate uniform fragment size and library yield. U.S. Pat. No. 9,683,230 and US Publication No. 2018-0155709, each of which are incorporated by reference herein in their entirety, describe uses of BLTs to control library fragment size.

Fragment size is a function of the ratio of transposomes to the amount and size of DNA and to the duration of the reaction. Even if these parameters are controlled in a solution-phase tagmentation reaction, however, size selection fractionation is commonly required as an additional step to remove excess small fragments. In other words, fragment size control can be better managed with BLTs as compared to solution-phase tagmentation.

In some embodiments, fBLTs allow for selection of final fragment size as a function of the spatial separation of the bound transposomes, independent of the quantity of transposome beads added to the tagmentation reaction. An additional limitation of solution-based tagmentation is that it is typically necessary to do some form of purification of the products of the tagmentation reaction both before and after PCR amplification. This typically necessitates some transfer of reactions from tube to tube. In contrast, tagmentation products on the fBLTs can be washed and later released for amplification or other downstream processing, thus avoiding the need for sample transfer. For example, in embodiments where transposomes are assembled on paramagnetic beads, purification of the tagmentation reaction products can easily be achieved by immobilizing the beads with a magnet and washing. Thus, in some embodiments, tagmentation and other downstream processing such as PCR amplification can all be performed in a single tube, vessel, droplet, or other container.

In some embodiments, the density of transposomes immobilized on the solid surface is selected to modulate fragment size and library yield of the immobilized fragments. In some embodiments, the spacing of active transposomes on the bead surface of a fBLT may be used to control the insert size distribution. For example, gaps on the bead surface may be filled with inactive transposomes (e.g., transposomes with inactive transposons).

### D. Mosaic End Removal

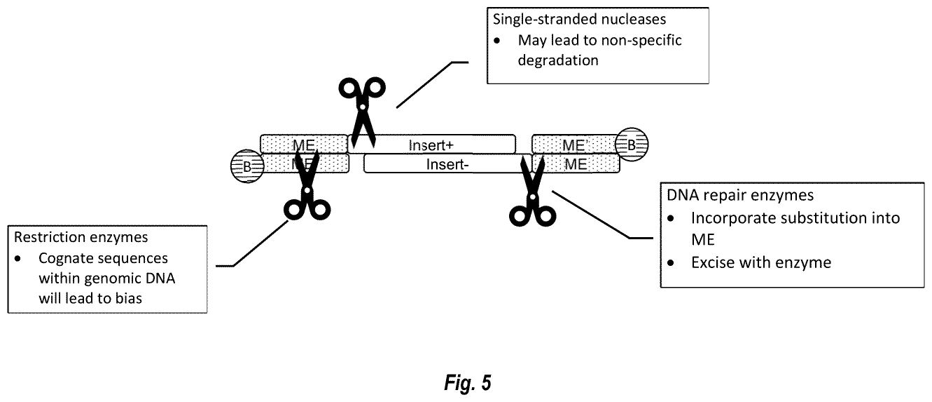

In order to enable transformation of Tn5 into a fragmentase system, a mechanism for selective removal of the mosaic end sequences after transposition is necessary. Such potential mechanisms could include (1) restriction enzymes that recognize a sequence within the Mosaic End, (2) single stranded DNAses that take advantage of the 9-nucleotide gap present on either side of the insert after transposition, and (3) either (a) an endonuclease or (b) a combination of a DNA glycosylase and heat, basic conditions, or an endonuclease/lyase that recognizes abasic sites (See FIG. 5). Restriction enzymes are disadvantageous because they would cleave at other cognate sites within the genomic DNA, leading to bias. Single stranded nucleases also could potentially be used to remove the mosaic end sequence. However, double stranded DNA is known to “breathe” at its ends, which often leads to off-target digestion of double stranded DNA and is difficult to control (See Neelam A. Desai and Vepatu Shankar, FEMS Microbiology Reviews 26(5):457-91 (2003)).

The present method with selective cleavage of the mosaic end using enzymes is a highly attractive mechanism for transforming Tn5 into a fragmentase system (i.e., to generate fragments lacking mosaic ends). As used herein, a “base modification” or “DNA base modification” refers to the position of a modified base (such as those described in Table 3) in a double-stranded nucleic acid that will be recognized by an enzyme (such as (a) an endonuclease or (b) a combination of a DNA glycosylase and heat, basic conditions, or an endonuclease/lyase that recognizes abasic sites), triggering cleavage at this modified base. In some embodiments, an endonuclease or DNA glycosylase is modification-specific.

In some embodiments, a base modification is cleaved using (1) an endonuclease or (2) a combination of a DNA glycosylase and heat, basic conditions, or an endonuclease/lyase that recognizes abasic sites. For example, a DNA glycosylase may produce an abasic site that is then acted upon by heat, basic conditions, or an endonuclease/lyase that recognizes abasic sites. USER reagents are an exemplary enzyme mix comprising a DNA glycosylase and an endonuclease/lyase that recognizes abasic sites. The user may choose how to cleave at an abasic site depending on their preferred workflow. FIG. 7B outlines how a modification-specific endonuclease can cleave a modified base in a 1-step reaction or a modification-specific glycosylase followed by an AP lyase/endonuclease or heat can cleave a modified base in a 2-step reaction.

Fragments prepared from such a transposition reaction followed by cleavage at a modified base will comprise inserts with 5′ overhangs with 5′ phosphate and 3′-OH, and 0-3 bases of ME sequence, depending on the site of modification at one or more of positions 16-19 of SEQ ID NO: 1.

In some embodiments, cleavage of the modified mosaic end sequence is mediated by (a) an endonuclease or (b) a combination of a DNA glycosylase and heat, basic conditions, or an endonuclease/lyase that recognizes abasic sites. In some embodiments, (a) an endonuclease or (b) a combination of a DNA glycosylase and heat, basic conditions, or an endonuclease/lyase that recognizes abasic sites can mediate cleavage at a uracil, an inosine, a ribose, an 8-oxoguanine, a thymine glycol, a modified purine, and/or a modified pyrimidine.

In some embodiments, the (a) an endonuclease or (b) a combination of a DNA glycosylase and heat, basic conditions, or an endonuclease/lyase that recognizes abasic sites is a USER, endonuclease V, RNAse HII, formamidopyrimidine-DNA glycosylase (FPG), oxoguanine glycosylase (OGG), endonuclease III (Nth), endonuclease VIII, a mixture of human alkyl adenine DNA glycosylase plus endonuclease VIII or endonuclease III, a mixture of and either thymine-DNA glycosylase (TDG) or mammalian DNA glycosylase-methyl-CpG binding domain protein 4 (MBD4) plus endonuclease VIII or endonuclease III, or DNA glycosylase/lyase ROS1 (ROS1). In some embodiments, ROSI can function as a modification-endonuclease based on its bifunctional glycosylase/lyase activity.

In some embodiments, the modified transposon end sequence comprises a uracil and the mixture is a N-glycosylase and an apurinic or apyrimidinic site (AP) lyase/endonuclease is a uracil-specific excision reagent (USER). In some embodiments, the USER is a mixture of uracil DNA glycosylase and endonuclease VIII or endonuclease III.

In some embodiments, the modified transposon end sequence comprises an inosine and the endonuclease is endonuclease V. In some embodiments, the modified transposon end sequence comprises a ribose and the endonuclease is RNAse HII.

In some embodiments, the modified transposon end sequence comprises a 8-oxoguanine and the endonuclease is formamidopyrimidine-DNA glycosylase (FPG) or oxoguanine glycosylase (OGG).

In some embodiments, the modified transposon end sequence comprises a thymine glycol and the DNA endonuclease is endonuclease III (Nth) or endonuclease VIII.

In some embodiments, the modified transposon end sequence comprises a modified purine and the DNA glycosylase and endonuclease/lyase that recognizes abasic sites is a mixture of human alkyl adenine DNA glycosylase (hAAG) plus endonuclease VIII or endonuclease III.

In some embodiments, the modified transposon end sequence comprises a modified pyrimidine and the DNA glycosylase is TDG or MBD4 and the endonuclease/lyase that recognizes abasic sites is endonuclease VIII or endonuclease III. An alternative modification-specific endonuclease for use with a modified transposon end comprising a modified pyrimidine is ROS1.

In some embodiments, a first transposon comprises a modified transposon end sequence comprising more than one mutation chosen from a uracil, an inosine, a ribose, an 8-oxoguanine, a thymine glycol, a modified purine, and/or a modified pyrimidine and the -and an endonuclease or DNA glycosylase and endonuclease/lyase that recognizes abasic sites are comprised in a mixture. In some embodiments, the endonuclease or DNA glycosylase and endonuclease/lyase that recognizes abasic sites mixture comprises more than enzyme chosen from a USER, endonuclease V, RNAse HII, formamidopyrimidine-DNA glycosylase (FPG), oxoguanine glycosylase (OGG), endonuclease III (Nth), endonuclease VIII, a mixture of hAAG plus endonuclease VIII/endonuclease III, or a mixture of TDG or MBD4 together with endonuclease VIII/endonuclease III, or ROS1. In some embodiments, methods with modified transposon end sequences comprising more than one mutation and an endonuclease and/or a combination of DNA glycosylase and endonuclease/lyase that recognizes abasic sites mixture improves the efficiency of cleavage of the mosaic end sequence as compared to methods with a modified transposon end sequences comprising a single mutation and a single endonuclease or combination of DNA glycosylase and endonuclease/lyase that recognizes abasic sites. For ROS1, a single endonuclease has both glycosylase and lyase function.

In some embodiments, a method of fragmenting a double-stranded nucleic acid comprises combining a sample comprising double-stranded nucleic acid with a transposome complex and preparing fragments.

In some embodiments, a method of preparing double-stranded nucleic acid fragments that lack all or part of the first transposon end comprises combining a sample comprising nucleic acid with transposome complexes and preparing fragments; and combining the sample with (1) an endonuclease or (2) a combination of a DNA glycosylase and heat, basic conditions, or an endonuclease/lyase that recognizes abasic sites and cleaving the first transposon end at the uracil, inosine, ribose, 8-oxoguanine, thymine glycol, a modified purine, and/or a modified pyrimidine within the mosaic sequence to remove all or part of the first transposon end from the fragments. In some embodiments, the modified purine is 3-methyladenine or 7-methylguanine. In some embodiments, the modified pyrimidine is 5-methylcytosine, 5-formylcytosine, or 5-carboxycytosine. In some embodiments, this method cleaves all or part of the first transposon end (the transferred strand) from the fragments.

In some embodiments, cleaving the first transposon end generates a sticky end for ligating an adapter. As used herein, a “sticky end” is an end of a double-stranded fragment wherein one strand is longer than the other (i.e., there is an overhang) and the overhang allows for ligation of an adapter comprising a complementary overhang.

In some embodiments, adapters are added after removing all or part of the first transposon end from fragments. In some embodiments, adapters are added by ligation. In some embodiments, end repair and A-tailing mixes enable ligation of adapters. One skilled in the art would be aware of other means to add adapters, such as PCR amplification or Click chemistry.

### E. Ligation of Adapters

In some embodiments, a method of preparing double-stranded nucleic acid fragments comprising adapters comprises combining a sample comprising nucleic acid with the transposome complexes described herein and preparing fragments; combining the sample with (1) an endonuclease or (2) a combination of a DNA glycosylase and heat, basic conditions, or an endonuclease/lyase that recognizes abasic sites and cleaving the first transposon end at the uracil, inosine, ribose, 8-oxoguanine, thymine glycol, modified purine, and/or a modified pyrimidine within the mosaic end sequence to remove all or part of the first transposon end from the fragments; and ligating an adapter onto the 5′ and/or 3′ ends of the fragments. A representative outline of steps is shown in FIG. 7A.

In some embodiments, adapters comprising sequence sequences are ligated onto library fragments after removal of all or part of the mosaic end sequence. Fragments that been subjected to ligation of an adapter to the 5′ and/or 3′ end of the fragment may be termed “tagged fragments.”

In some embodiments, the ligating is performed with a DNA ligase.

In some embodiments, the adapter comprises a double-stranded adapter.

In some embodiments, adapters are added to the 5′ and 3′ end of fragments. In some embodiments, the adapters added to the 5′ and 3′ end of the fragments are different.

A wide variety of library preparation methods comprising a step of adapter ligation are known in the art, such as TruSeq and TruSight Oncology 500 (See, for example, TruSeq® RNA Sample Preparation v2 Guide, 15026495 Rev. F, Illumina, 2014). Adapters used with other ligation methods may be used in the present method (See, for example, Illumina Adapter Sequences, Illumina, 2021). Adapters for use in the present invention also include those described in WO 2008/093098, WO 2008/096146, WO 2018/208699, and WO 2019/055715, which are each incorporated by reference in their entirety herein.

In some embodiments, adapter ligation may allow for more flexible incorporation of adapters (such as adapters with longer lengths) as compared to methods of tagging fragments via tagmentation (wherein adapter sequences are incorporated into fragments during the transposition reaction). In some methods involving tagmentation, additional adapter sequences may be incorporated by PCR reactions (such as those described in US Patent Publication No. 20180201992A1), and the present methods may obviate the need for an additional PCR step to incorporate additional adapter sequences.

Ligation technology is commonly used to prepare NGS libraries for sequencing. In some embodiments, the ligation step uses an enzyme to connect specialized adapters to both ends of DNA fragments. In some embodiments, an A-base is added to blunt ends of each strand, preparing them for ligation to the sequencing adapters. In some embodiments, each adapter contains a T-base overhang, providing a complementary overhang for ligating the adapter to the A-tailed fragmented DNA.

Adapter ligation protocols are known to have advantages over other methods. For example, adapter ligation can be used to generate the full complement of sequencing primer hybridization sites for single, paired-end, and indexed reads. In some embodiments, adapter ligation eliminates a need for additional PCR steps to add the index tag and index primer sites.

In some embodiments, the adapter comprises a unique molecular identifier (UMI), primer sequence, anchor sequence, universal sequence, spacer region, index sequence, capture sequence, barcode sequence, cleavage sequence, sequencing-related sequence, and combinations thereof. As used herein, a “barcode sequence” refers to a sequence that may be used to differentiate samples. As used herein, a sequencing-related sequence may be any sequence related to a later sequencing step. A sequencing-related sequence may work to simplify downstream sequencing steps. For example, a sequencing-related sequence may be a sequence that would otherwise be incorporated via a step of ligating an adapter to nucleic acid fragments. In some embodiments, the adapter sequence comprises a P5 or P7 sequence (or their complement) to facilitate binding to a flow cell in certain sequencing methods.

In some embodiments, the adapter comprises a UMI. In some embodiments, an adapter comprising a UMI is ligated to both the 3′ and 5′ end of fragments.

In some embodiments, the adapter may be a forked adapter. As used herein, a “forked adapter” refers to an adapter comprising two strands of nucleic acid, wherein the two strands each comprise a region that is complementary to the other strand and a region that is not complementary to the other strand. In some embodiments, the two strands of nucleic acid in the forked adapter are annealed together before ligation, with the annealing based on complementary regions. In some embodiments, the complementary regions each comprise 12 nucleotides. In some embodiments, a forked adapter is ligated to both strands at the end of a double-stranded DNA fragment. In some embodiments, a forked adapter is ligated to one end of a double-stranded DNA fragment. In some embodiments, a forked adapter is ligated to both ends of a double-stranded DNA fragment. In some embodiments, the forked adapters on opposite ends of a fragment are different. In some embodiments, one strand of the forked adapter is phosphorylated at its 5′ end to promote ligation to fragments. In some embodiments, one strand of the forked adapter has a phosphorothioate bond directly before a 3′ T. In some embodiments, the 3′ T is an overhang (i.e., not paired with a nucleotide in the other strand of the forked adapter). In some embodiments, the 3′ T overhang can basepair with an A-tail present on a library fragment. In some embodiments, the phosphorothioate bond blocks exonuclease digestion of the 3′ T overhang. In some embodiments, PCR with partially complementary primers is used after adapter ligation to extend ends and resolve the forks.

In some embodiments, an adapter may comprise a tag. The terms “tag” as used herein refers to a portion or domain of a polynucleotide that exhibits a sequence for a desired intended purpose or application. Tag domains can comprise any sequence provided for any desired purpose. For example, in some embodiments, a tag domain comprises one or more restriction endonuclease recognition sites. In some embodiments, a tag domain comprises one or more regions suitable for hybridization with a primer for a cluster amplification reaction. In some embodiments, a tag domain comprises one or more regions suitable for hybridization with a primer for a sequencing reaction. It will be appreciated that any other suitable feature can be incorporated into a tag domain. In some embodiments, the tag domain comprises a sequence having a length from 5 bp to 200 bp. In some embodiments, the tag domain comprises a sequence having a length from 10 bp to 100 bp. In some embodiments, the tag domain comprises a sequence having a length from 20 bp to 50 bp. In some embodiments, the tag domain comprises a sequence having a length of 5, 6, 7, 8, 9, 10, 20, 30, 40, 50, 60, 70, 80, 90, 100, 150 or 200 bp.

The tag can include one or more functional sequences or components (e.g., primer sequences, anchor sequences, universal sequences, spacer regions, or index tag sequences) as needed or desired.

In some embodiments, the tag comprises a region for cluster amplification. In some embodiments, the tag comprises a region for priming a sequencing reaction.

In some embodiments, the method further comprises amplifying the fragments on the solid support by reacting a polymerase and an amplification primer corresponding to a portion of a tag. In some embodiments, a portion of the adapter ligated onto fragments after removal of all or part of the mosaic end sequence comprises an amplification primer. In some embodiments, the tag of the first transposon comprises an amplification primer.

In some embodiments a tag comprises an A14 primer sequence. In some embodiments, a tag comprises a B15 primer sequence.

In some embodiments, transposomes on an individual bead carry a unique index, and if a multitude of such indexed beads are employed, phased transcripts will result.

Adapters that are ligated onto library fragments can have advantages over adapters that are incorporated during tagmentation. For example, unique molecular identifiers (UMIs) can be used to enable high-sensitivity variant detection by labeling single fragments with unique sequence tags prior to PCR (See Jesse J. Salk, et al., Nature Reviews Genetics 19(5): 269-85 (2018)). Some library preparation products, such as TSO 500 (Illumina), include a ligation-based UMI offering in which the UMI sequence is incorporated adjacent to the library insert, enabling simultaneous sequencing as a part of the insert read. Therefore, development of fBLTs enables existing ligation-based products to be leveraged (such as use of existing adapters and protocols), while simultaneously enabling compatibility with existing enrichment workflows and onboard sequencing primers.

FIG. 19 presents some representative different adapter workflows that a user may wish to employ with fBLTs. For example, a high-sensitivity UMI workflow may be used, wherein adapters incorporate UMIs. Alternatively, a PCR workflow that adds UMIs during PCR amplification may be used with standard forked adapters. In addition, a PCR-free workflow may be used with indexes forked adapters that avoid the need for PCR. Accordingly, an advantage of fBLTs is that they allow the user to choose adapters of highest interest for their particular library preparation. Other library preparation methods, such as tagmentation, have greater stringencies in the composition of adapter sequences that can be used.

### F. Samples and Target Nucleic Acids

In some embodiments, a sample comprises nucleic acid. The nucleic acid comprised in a sample may be referred to as “target nucleic acid.” In some embodiments, the sample comprises DNA. In some embodiments, the DNA is genomic DNA. In some embodiments, the target nucleic acid is double-stranded DNA.

In some embodiments, the sample comprises RNA. In some embodiments, the RNA may be converted to double-stranded cDNA or to DNA:RNA duplexes (i.e. RNA hybridized to a single strand of cDNA).

In some embodiments, the nucleic acid is double-stranded DNA. In some embodiments, the nucleic acid is RNA, and double-stranded cDNA or DNA:RNA duplexes are generated before combining with the transposome complexes.

The biological sample can be any type that comprises nucleic acid. For example, the sample can comprise nucleic acid in a variety of states of purification, including purified nucleic acid. However, the sample need not be completely purified, and can comprise, for example, nucleic acid mixed with protein, other nucleic acid species, other cellular components, and/or any other contaminant. In some embodiments, the biological sample comprises a mixture of nucleic acid, protein, other nucleic acid species, other cellular components, and/or any other contaminant present in approximately the same proportion as found in vivo. For example, in some embodiments, the components are found in the same proportion as found in an intact cell. In some embodiments, the biological sample has a 260/280 absorbance ratio of less than or equal to 2.0, 1.9, 1.8, 1.7, 1.6, 1.5, 1.4, 1.3, 1.2, 1.1, 1.0, 0.9, 0.8, 0.7, or 0.60. In some embodiments, the biological sample has a 260/280 absorbance ratio of at least 2.0, 1.9, 1.8, 1.7, 1.6, 1.5, 1.4, 1.3, 1.2, 1.1, 1.0, 0.9, 0.8, 0.7, or 0.60. Because the methods provided herein allow nucleic acid to be bound to solid supports, other contaminants can be removed merely by washing the solid support after surface bound transposition occurs. The biological sample can comprise, for example, a crude cell lysate or whole cells. For example, a crude cell lysate that is applied to a solid support in a method set forth herein, need not have been subjected to one or more of the separation steps that are traditionally used to isolate nucleic acids from other cellular components. Exemplary separation steps are set forth in Maniatis et al., Molecular Cloning: A Laboratory Manual, 2d Edition, 1989, and Short Protocols in Molecular Biology, ed. Ausubel, et al, hereby incorporated by reference.

In some embodiments, the sample that is applied to the solid support has a 260/280 absorbance ratio that is less than or equal to 1.7.

Thus, in some embodiments, the biological sample can comprise, for example, blood, plasma, serum, lymph, mucus, sputum, urine, semen, cerebrospinal fluid, bronchial aspirate, feces, and macerated tissue, or a lysate thereof, or any other biological specimen comprising nucleic acid.

In some embodiments, the sample is blood. In some embodiments, the sample is a cell lysate. In some embodiments, the cell lysate is a crude cell lysate. In some embodiments, the method further comprises lysing cells in the sample after applying the sample to a solid support to generate a cell lysate.

In some embodiments, the sample is a biopsy sample. In some embodiments, the biopsy sample is a liquid or solid sample. In some embodiments, a biopsy sample from a cancer patient is used to evaluate sequences of interest to determine if the subject has certain mutations or variants in predictive genes.

One advantage of the methods and compositions presented herein that a biological sample can be added to a flow cell and subsequent lysis and purification steps can all occur in the flow cell without further transfer or handling steps, simply by flowing the necessary reagents into the flow cell.

### G. Gap-Fill Ligation, Phosphorylating, and A-Tailing

In some embodiments, gaps in the DNA sequence left after the transposition event can also be filled in using a strand displacement extension reaction, such one comprising a Bst DNA polymerase and dNTP mix. In some embodiments, a gap-fill ligation is performed using an extension-ligation mix buffer.

In some embodiments, a method comprises treating the plurality of 5′ fragments with a polymerase and a ligase to extend and ligate the strands to produce fully double-stranded fragments.

The library of double-stranded DNA fragments can then optionally be amplified (such as with cluster amplification) and sequenced with a sequencing primer.

In some embodiments, the all or part of the first transposon end that is cleaved is partitioned away from the rest of the sample.

In some embodiments, the method further comprises filling in the 3′ ends of the fragments and phosphorylating the 3′ ends of fragments with a kinase before ligating. In some embodiments, the ends generated by cleavage of the mosaic end sequence are not blunt (i.e., one strand of the double-stranded fragment has a sticky overhang as compared to the other end). In some embodiments, a sticky overhang is not filled in and an adapter is ligated onto the sticky overhang, wherein the adapter has a complementary sticky end.

In some embodiments, the fragments comprise 0-3 bases of the mosaic end sequence. In some embodiments, a strand of a double-stranded fragment has a different number of bases from the mosaic end sequence as compared to the other strand (i.e., the end of the fragment has an overhang and is not a blunt end). In some embodiments, the overhang generated by cleavage of the mosaic end sequence is filled-in. In some embodiments, filling in of ends generated by cleavage of the mosaic end sequence is performed with T4 DNA polymerase.

In some embodiments, the method further comprises adding a single A overhang to the 3′ end of the fragments. In some embodiments, adding single A overhang may be referred to as “A-tailing.” In some embodiments, A-tailing improves ligation of an adapter, such as a forked adapter. In some embodiments, one strand of a forked adapter comprises a T overhang that can base pair with the A-tail on a fragment.

In some embodiments, a polymerase adds the single A overhang. In some embodiments, the polymerase is (i) Taq or (ii) Klenow fragment, exo-.

### H. Amplification

The present disclosure further relates to amplification of fragments produced according to the methods provided herein. In some embodiments, the fragments are tagged by ligation of an adapter or at one both ends of the fragments. In some embodiments, immobilized fragments are amplified on a solid support. In some embodiments, the solid support is the same solid support upon which the surface bound transposition occurs. In such embodiments, the methods and compositions provided herein allow sample preparation to proceed on the same solid support from the initial sample introduction step through amplification and optionally through a sequencing step.

In some embodiments, fragments are amplified before sequencing.

For example, in some embodiments, immobilized fragments are amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to herein as “clustered arrays”. The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, in some embodiments via a covalent attachment. Cluster amplification methodologies are examples of methods wherein an immobilized nucleic acid template is used to produce immobilized amplicons. Other suitable methodologies can also be used to produce immobilized amplicons from immobilized DNA fragments produced according to the methods provided herein. For example, one or more clusters or colonies can be formed via solid-phase PCR whether one or both primers of each pair of amplification primers are immobilized.

In other embodiments, fragments are amplified in solution. For example, in some embodiments, fragments are cleaved or otherwise liberated from the solid support and amplification primers are then hybridized in solution to the liberated molecules. In other embodiments, amplification primers are hybridized to tagged fragments for one or more initial amplification steps, followed by subsequent amplification steps in solution. In some embodiments, an immobilized nucleic acid template can be used to produce solution-phase amplicons.

It will be appreciated that any of the amplification methodologies described herein or generally known in the art can be utilized with universal or target-specific primers to amplify tagged fragments. Suitable methods for amplification include, but are not limited to, the polymerase chain reaction (PCR), strand displacement amplification (SDA), transcription mediated amplification (TMA) and nucleic acid sequence-based amplification (NASBA), as described in U.S. Pat. No. 8,003,354, which is incorporated herein by reference in its entirety. The above amplification methods can be employed to amplify one or more nucleic acids of interest. For example, PCR, including multiplex PCR, SDA, TMA, NASBA and the like can be utilized to amplify immobilized DNA fragments. In some embodiments, primers directed specifically to the nucleic acid of interest are included in the amplification reaction.

Other suitable methods for amplification of nucleic acids can include oligonucleotide extension and ligation, rolling circle amplification (RCA) (Lizardi et al., Nat. Genet. 19:225-232 (1998), which is incorporated herein by reference) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835, all of which are incorporated by reference) technologies. It will be appreciated that these amplification methodologies can be designed to amplify immobilized DNA fragments. For example, in some embodiments, the amplification method can include ligation probe amplification or oligonucleotide ligation assay (OLA) reactions that contain primers directed specifically to the nucleic acid of interest. In some embodiments, the amplification method can include a primer extension-ligation reaction that contains primers directed specifically to the nucleic acid of interest. As a non-limiting example of primer extension and ligation primers that can be specifically designed to amplify a nucleic acid of interest, the amplification can include primers used for the GoldenGate assay (Illumina, Inc., San Diego, CA) as exemplified by U.S. Pat. Nos. 7,582,420 and 7,611,869, each of which is incorporated herein by reference in its entirety.

Exemplary isothermal amplification methods that can be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587, each of which is incorporated herein by reference in its entirety. Other non-PCR-based methods that can be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyperbranched strand displacement amplification which is described in, for example Lage et al., Genome Research 13:294-307 (2003), each of which is incorporated herein by reference in its entirety. Isothermal amplification methods can be used with the strand-displacing Phi 29 polymerase or Bst DNA polymerase large fragment, 5′→3′ exo- for random primer amplification of genomic DNA. The use of these polymerases takes advantage of their high processivity and strand displacing activity. High processivity allows the polymerases to produce fragments that are 10-20 kb in length. As set forth above, smaller fragments can be produced under isothermal conditions using polymerases having low processivity and strand-displacing activity such as Klenow polymerase. Additional description of amplification reactions, conditions and components are set forth in detail in the disclosure of U.S. Pat. No. 7,670,810, which is incorporated herein by reference in its entirety.

Another nucleic acid amplification method that is useful in the present disclosure is Tagged PCR which uses a population of two-domain primers having a constant 5′ region followed by a random 3′ region as described, for example, in Grothues et al. Nucleic Acids Res. 21(5):1321-2 (1993), incorporated herein by reference in its entirety. The first rounds of amplification are carried out to allow a multitude of initiations on heat denatured DNA based on individual hybridization from the randomly synthesized 3′ region. Due to the nature of the 3′ region, the sites of initiation are contemplated to be random throughout the genome. Thereafter, the unbound primers can be removed, and further replication can take place using primers complementary to the constant 5′ region.

### I. Sequencing

In some embodiments, the method further comprises sequencing the fragments after removing all or part of the first transposon end from the fragment.

In some embodiments, the method further comprises sequencing the fragments after ligating the adapter. In some embodiments, the method does not require amplification of fragments before sequencing. In some embodiments, fragments are amplified before sequencing.

In some embodiments, the method further comprises enriching fragments of interest after ligating the adapter and before sequencing. Enrichment may be performed with a variety of commercially available reagents, such as RNA Prep with Enrichment Reference Guide (Illumina Document No: 1000000124435).

The present disclosure further relates to sequencing of tagged fragments produced according to the methods provided herein. In some embodiments, a method comprises sequencing one or more of the 5′ tagged and/or 3′ tagged fragments or fully double-stranded tagged fragments after ligation of an adapter at one or both ends of the fragments. In some embodiments, the adapter comprises a sequence primer binding sequence to facilitate the sequencing.

The tagged fragments can be sequenced according to any suitable sequencing methodology, such as direct sequencing, including sequencing by synthesis, sequencing by ligation, sequencing by hybridization, nanopore sequencing and the like. In some embodiments, the tagged fragments are sequenced on a solid support. In some embodiments, the solid support for sequencing is the same solid support upon which ligation of adapters occurs. In some embodiments, the solid support for sequencing is the same solid support upon which the amplification occurs.

One exemplary sequencing methodology is sequencing-by-synthesis (SBS). In SBS, extension of a nucleic acid primer along a nucleic acid template (e.g. a target nucleic acid or amplicon thereof) is monitored to determine the sequence of nucleotides in the template. The underlying chemical process can be polymerization (e.g. as catalyzed by a polymerase enzyme). In a particular polymerase-based SBS embodiment, fluorescently labeled nucleotides are added to a primer (thereby extending the primer) in a template dependent fashion such that detection of the order and type of nucleotides added to the primer can be used to determine the sequence of the template.

Flow cells provide a convenient solid support for housing amplified DNA fragments produced by the methods of the present disclosure. One or more amplified DNA fragments in such a format can be subjected to an SBS or other detection technique that involves repeated delivery of reagents in cycles. For example, to initiate a first SBS cycle, one or more labeled nucleotides, DNA polymerase, etc., can be flowed into/through a flow cell that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be adapted for application of pyrosequencing to amplicons produced according to the present disclosure are described, for example, in WIPO Pat. App. Pub. No. WO 2012058096, US 2005/0191698 A1, U.S. Pat . Nos. 7,595,883, and 7,244,559, each of which is incorporated herein by reference.

Some embodiments can utilize methods involving the real-time monitoring of DNA polymerase activity. For example, nucleotide incorporations can be detected through fluorescence resonance energy transfer (FRET) interactions between a fluorophore-bearing polymerase and y-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

Another useful sequencing technique is nanopore sequencing (see, for example, Deamer et al. Trends Biotechnol. 18, 147-151 (2000); Deamer et al. Acc. Chem. Res. 35:817-825 (2002); Li et al. Nat. Mater. 2:611-615 (2003), the disclosures of which are incorporated herein by reference). In some nanopore embodiments, the target nucleic acid or individual nucleotides removed from a target nucleic acid pass through a nanopore. As the nucleic acid or nucleotide passes through the nanopore, each nucleotide type can be identified by measuring fluctuations in the electrical conductance of the pore. (U.S. Pat. No. 7,001,792; Soni et al. Clin. Chem. 53, 1996-2001 (2007); Healy, Nanomed. 2, 459-481 (2007); Cockroft et al. J. Am. Chem. Soc. 130, 818-820 (2008), the disclosures of which are incorporated herein by reference).

Exemplary methods for array-based expression and genotyping analysis that can be applied to detection according to the present disclosure are described in U.S. Pat. Nos. 7,582,420; 6,890,741; 6,913,884 or 6,355,431 or US Pat. Pub. Nos. 2005/0053980 A1; 2009/0186349 A1 or US 2005/0181440 A1, each of which is incorporated herein by reference.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines, and the like. A flow cell can be configured and/or used in an integrated system for detection of target nucleic acids. Exemplary flow cells are described, for example, in US 2010/0111768 A1 and US Pub. No. 2012/0270305 A1, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in US Pub. No. 2012/0270305, which is incorporated herein by reference.

### EXAMPLES

### Example 1. Representative Method of Library Preparation Using fBLTs

A method using Tn5 with fBLTs may include the following steps.
1. Tn5 enzyme is complexed with a mutated mosaic end (ME) transposon containing an encoded DNA base modification (e.g. uracil, 8-oxoG, etc.) near the 3′ end of the transferred strand. If desired, the transposon DNA can be biotinylated to facilitate formation of bead-linked transposomes (BLTs, in this case fBLTs).
2. The resulting transposome is used to fragment input DNA, such as genomic DNA.
3. The resulting fragmented DNA is treated with an appropriate enzyme that is (1) an endonuclease or (2) a combination of a DNA glycosylase and heat, basic conditions, or an endonuclease/lyase that recognizes abasic sites (e.g., USER or Fpg), resulting in cleavage of the transferred strand. Depending on the site of the modified base and the identity of the enzyme(s) for cleavage, some bases of the mosaic end may remain attached to the library fragments.
4. A DNA polymerase is used to fill in the 3′ ends of the library fragments. Depending on the enzyme used, a kinase may also be necessary to ensure proper phosphorylation for ligation.
5. An A-tailing polymerase (e.g. Taq, Klenow exo-) is used to add a single A overhang to the 3′ end of library fragments
6. Appropriate library adapters are ligated to the library inserts with a DNA ligase.

In this way, library fragments can be generated using transposition, while the adapters are added to the library fragments by ligation. This method allows for removal of all or part of the first transposon end from fragments before the ligation of an adapter. In some embodiments, the all or part of the first transposon end is partitioned from the rest of the sample before the ligating.

Other modifications of this protocol may be possible; for example, alternative strategies, such as chemical approaches, can be used to cleave the mosaic end selectively. Furthermore, a short sequence of remaining “mosaic end” can potentially be used to facilitate a robust “sticky end” ligation of >1 bp as an alternative to A-tailing of sample DNA and relying on weaker hybridization of a single base overhang between sample and adapter prior to ligation.

### Example 2. Mutational Analysis of Mosaic End with Tn5v3

A mutational screening experiment, with a focus on the 4 base pairs at the 3′ end of the transferred strand, was carried out. A FRET activity assay was employed to measure the activity of Tn5v3 with modified transposon ends comprising mutated mosaic end sequences.

As shown in FIG. 6A, Tn5v3 was able to recognize a variety of canonical mutations within the mosaic end sequence, with only a modest decrease in activity. Even mosaic ends with multiple mutations were tolerated, albeit with approximately 2-fold loss in activity. Interestingly, an A18C mutation resulted in poor activity, but activity was rescued when the mutated transferred strand was annealed to a wild-type non-transferred strand.

Having demonstrated that Tn5v3 could tolerate canonical mutations within the mosaic end, specifically at positions proximal to the 3′ end of the transferred strand, it was investigated whether modified bases, such as those listed in Table 3, could also be tolerated. Transposons were prepared with a single uracil, inosine, or ribose and assayed using the FRET activity assay. All of the tested modified base-containing MEs were tolerated by Tn5, albeit with a modest decrease in activity (FIG. 6B).

### Example 3. Library Preparation and Sequencing Using Transposition-Ligation

Building upon the finding that uracil, inosine, and ribose modifications of the ME transferred strand were well tolerated by Tn5v3, library preparation using a fragmentase-Tn5 approach was attempted using soluble transposomes (also known as solution-phase transposomes, such as the method outlined in FIG. 7A). Transposomes bearing modified bases were incubated with 1 ng Lambda DNA (NEB N3011S), based on the protocols available for soluble tagmentation, such as those described for the Illumina DNA Nextera® XT DNA Library Preparation Kit (see Nextera XT Reference Guide, Document 770-2012-011). Subsequently, DNA libraries were treated with the appropriate enzyme, i.e., (1) an endonuclease or (2) a combination of a DNA glycosylase and heat, basic conditions, or an endonuclease/lyase that recognizes abasic sites, followed by T4 DNA polymerase to fill in the 3′ ends of the library fragments. Libraries were then treated with Illumina A-tailing and Ligation mixes to ligate UMI-containing forked adapters. Following PCR amplification, the resulting libraries were analyzed via Bioanalyzer. All three modified base-enzyme pairs resulted in the formation of library, with USER showing highest conversion, evident through the fragment peak between 300-400 bp (FIG. 7C).

### Example 4: Comparison of USER-Fragmentase Libraries With Alternative Modification Sites

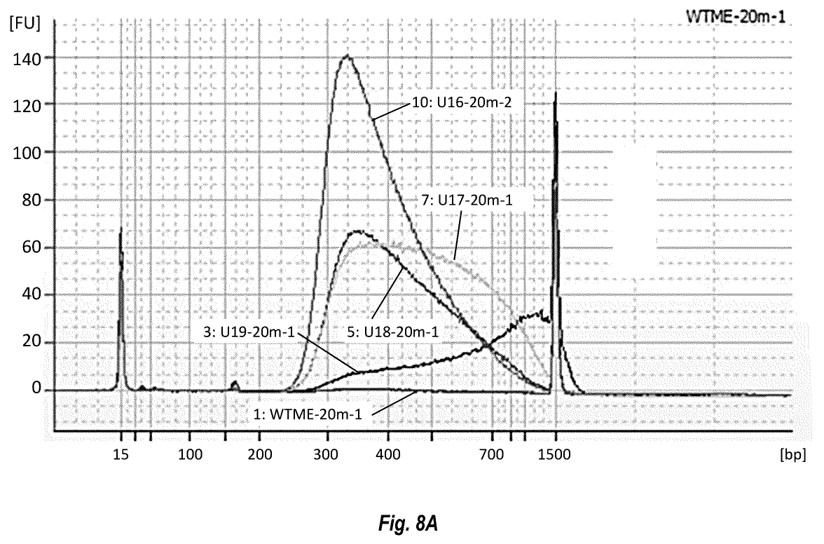

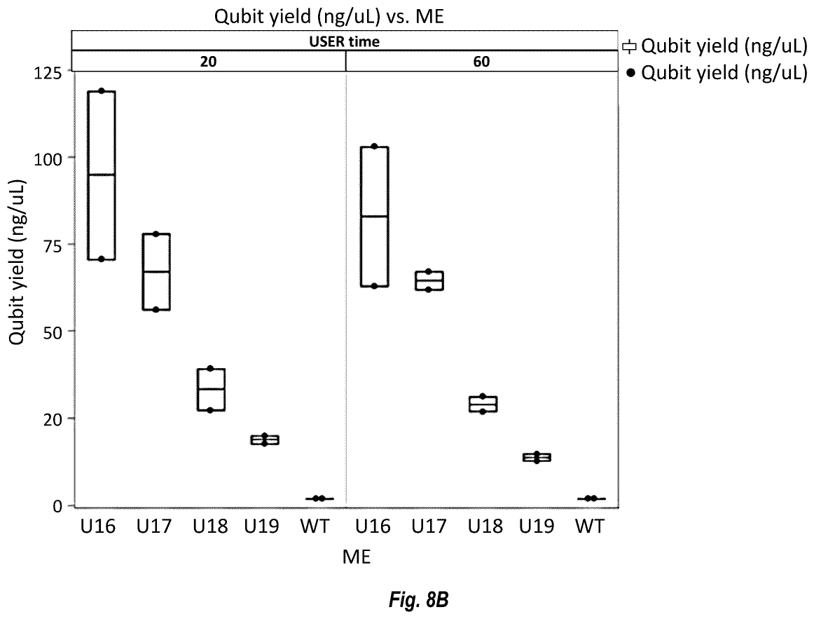

The uracil-USER pair (i.e., uracil substitution in the mosaic end sequence and USER as enzyme for cleavage of mosaic end sequence after transposition) was selected for further characterization of the fragmentase library preparation workflow. Transposomes with U16, U17, U18, and U19 modifications in the mosaic end were tested, alongside a wild-type (WT) mosaic end (SEQ ID NO: 1). Electrophoretic analysis of the resulting libraries showed that the fragment size distribution differs based on the site of modification (FIG. 8A). Furthermore, Qubit quantification of library yield showed that the yield was highest for the U16 ME, and yield decreased as the uracil modification was moved closer to the 3′ end (FIG. 8B). Transposomes were normalized by FRET activity for use in library preparation, so it is possible that these differences are due to variability in USER-recognition of these alternative ME substrates. Importantly, the wild-type transposome does not result in the formation of libraries, suggesting that cleavage of the mosaic end transferred strand is necessary to generate library fragments compatible with ligation, likely due to the gap-filling step with T4 DNA polymerase. In other words, adapters do not ligate onto library fragments, unless the mosaic end sequence is cleaved.

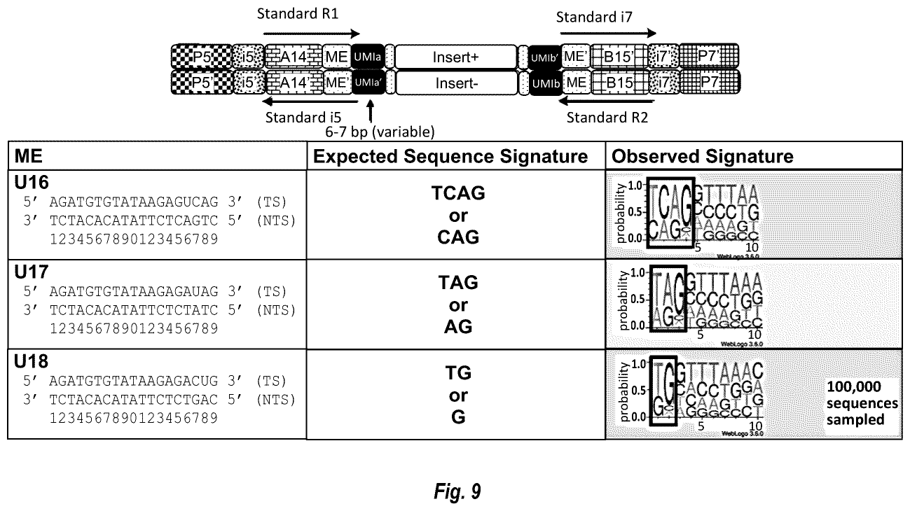

The USER enzyme mix acts by excising the uracil base, and thus for these modification sites, one would expect that 0-3 bases of the mosaic end will remain in the resulting libraries. After sequencing U16, U17, and U18 libraries, evidence of this ME “scar” adjacent to the library insert was assessed. The UMI ligation adapters used in this study contain a variable 6-7 basepair UMI sequence adjacent to the “T” overhang, and thus a distribution of library fragments that are shifted by 1 basepair was expected. Sampling of 100,000 sequences from each library type showed the expected sequence signature for each ME modification site (FIG. 9).

### Example 5. Fragmentase Bead-Linked Transposomes (fBLT)

Bead-linked transposomes are typically prepared by biotinylation of transposon DNA, which enables binding of the resulting transposomes to streptavidin beads. Initial efforts to immobilize the U16 transposome resulted in significantly lower BLT activity than expected (data not shown). Based on this finding, a mixed transposon consisting of the U16-transferred strand and wild-type non-transferred strand was used for pilot studies due to improved performance on BLTs. These fBLTs were loaded at a transposome density of 66 active unit/μL (AU/μL) to achieve similar library fragment distribution as enrichment BLTs (eBLTs) used in Illumina DNA Prep for Enrichment.

A preliminary study was conducted to assess the feasibility of fBLT-based library prep. Input DNA consisted of a mixture of NA12877 and NA12878 human gDNA and SspI-linearized phiX DNA at equimolar concentrations (approximately 15,000 genome copies each, 50 ng human gDNA equivalent).

Fragmentase-BLT libraries were prepared using a streamlined workflow in which USER cleavage, end repair, and A-tailing steps are combined (FIG. 10A). For comparison, libraries were also prepared with eBLTs according to the protocols set forth in the DNA Prep with Enrichment Reference Guide (1000000048041). Resulting fBLT libraries had a median library yield of approximately 300 ng and a similar fragment size distribution compared to eBLT libraries (FIGS. 10B and 10C). The slightly larger fragment size of the fBLT libraries may potentially be attributed to the 0.8×SPRI that was employed after adapter ligation.

Following library preparation, libraries were enriched following the enrichment protocols set forth in the RNA Prep with Enrichment Reference Guide (Illumina Document No: 1000000124435), using a combined panel consisting of TruSight Cancer and a custom panel targeting the whole genome of PhiX. Libraries were sequenced and FASTQ files were trimmed to remove the UMI sequence from fBLT samples. Comparative analyses of fBLT and eBLT were performed without UMIs using Dragen Enrichment v3.7.5 to characterize performance with the TruSight Cancer panel. Duplicate samples, consisting of 10% NA12877 in a background of NA12878, were analyzed for each library type. fBLT libraries showed similar performance to eBLT libraries, albeit with lower mean target coverage depth (Table 4). fBLT performance has the potential to be improved through workflow and BLT optimization.

TABLE 4
Summarized sequencing metrics for eBLT and fBLT samples

| | eBLT | fBLT |
|---|---|---|
| Mean target coverage depth | 1747 | 665 |
| Uniformity of coverage | 99% | 99% |
| Padded read enrichment | 75% | 75% |
| Sensitivity | 98.0% | 99.0% |
| Specificity | 99.997% | 99.997% |

In Table 4, data are reported as the mean of two replicates. Samples included a 10% spike of NA12877 gDNA into a background of NA12878 gDNA and were enriched using the TruSight Cancer panel.

This method allows an enzymatic approach for the fragmentation of DNA samples for NGS library preparation, wherein the resulting fragments are available for ligation of adapters. A benefit to users is eliminating the need to purchase expensive sonicators as capital equipment and gain the ease and speed of using a high-throughput enzymatic method for fragmentation of sample nucleic acids. The fBLT technology leverages the unique advantages of BLT technology and extends its compatibility to include and re-use a variety of ligation-based approaches. A key innovation enabling this advance is the incorporation of mutations into the mosaic end sequence to produce modified bases, which allows for site-specific cleavage of the transferred first transposon end while maintaining recognition by Tn5. By decoupling the enzymatic fragmentation and adapter tagging steps in the library preparation protocol, the addition of features such as forked adapters, barcodes, and UMIs can be enabled, while retaining compatibility with standard sequencing methods. Based on these unique advantages, fBLTs could be employed in a variety of applications such as UMI library preparation and PCR-free library preparation.

### Example 6. Optimization of Conditions for Methods With fBLTs

A variety of fBLTs comprising different modified transposon ends were examined. The different modified transposon ends comprised substitutions at positions A16, C17, A18, or G19 within the mosaic end sequence in comparison to SEQ ID NO: 1.

Bead-linked transposomes bearing modified mosaic ends (fBLTs) were incubated with 10-50 ng human sample DNA based on the protocols available for BLT tagmentation, such as those described for the Illumina DNA Prep with Enrichment Library Preparation kit (see Illumina DNA Prep with Enrichment reference guide, document 1000000048041). Subsequently, DNA libraries were treated with the appropriate DNA endonuclease to cleave the mosaic end. Samples were then subjected to a ligation-based library preparation workflow. Fragmented sample DNA was treated with Illumina end repair, A-tailing, and ligation reagents to enable adapter ligation.

Results on library conversion with different fBLTs are shown in FIG. 12. Experiments were performed to directly assess BLT activity without requiring downstream sequencing. Inosine, oxo-guanine, and uracil mutations at position A16 all led to lower BLT activity as compared to the same mutations at positions C17, A18, and G19. Thus, while modifications at position A16 of SEQ ID NO: 1 were well-tolerated in soluble transposomes, modifications at other positions yielded higher BLT activity. Therefore, transposon ends with modifications at position A16 may be of higher value for methods with soluble transposomes.

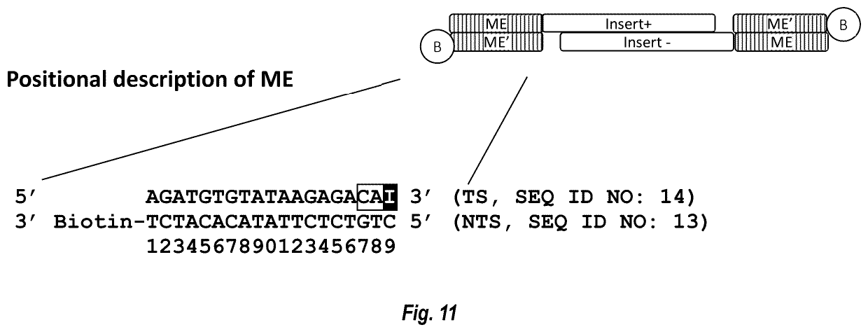

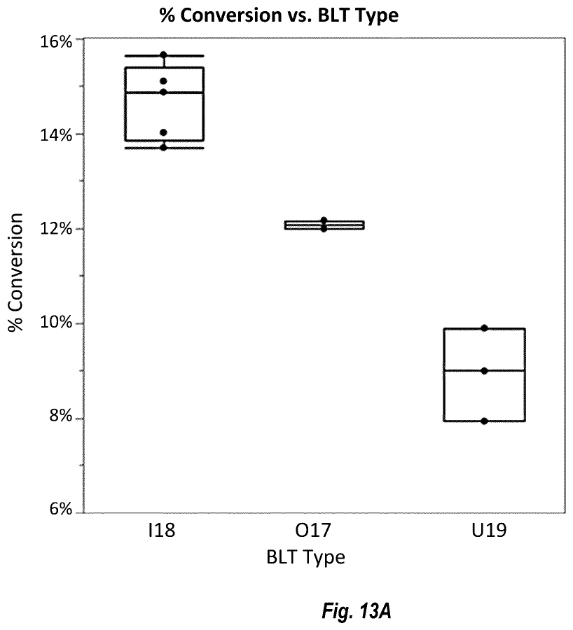

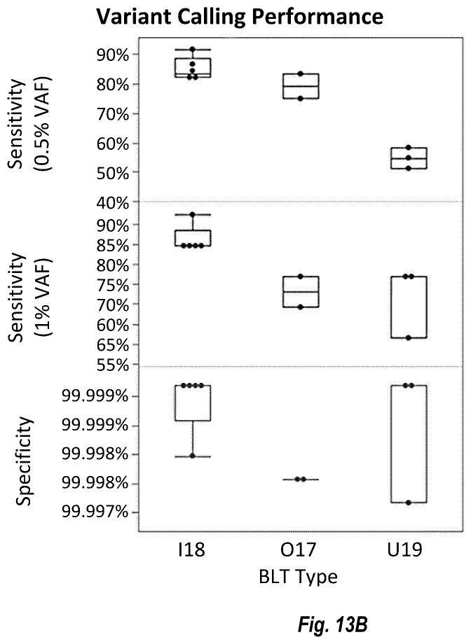

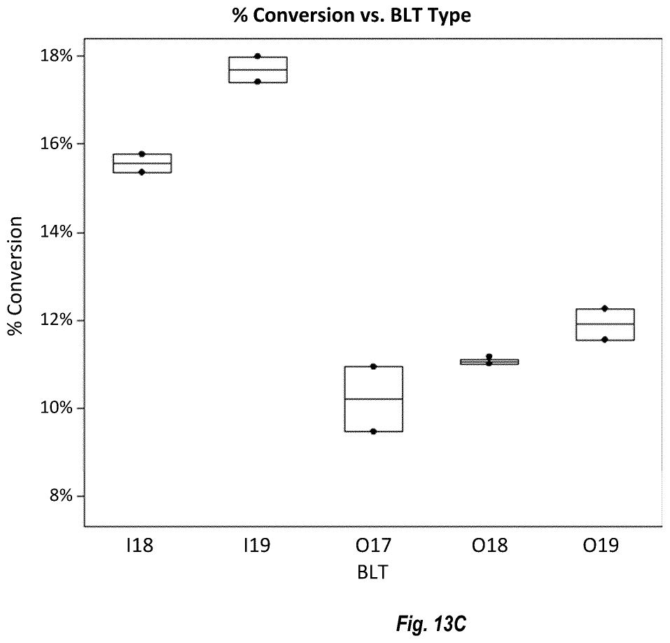

A variety of different fBLTs with inosine, oxo-guanine, or uracil mutations at positions C17, A18, and G19 were then assessed, as shown in FIGS. 13A-13C. Data showed that inosine modifications had the best performance as measured by library conversion efficiency and variant calling metrics. In particular, G19I (I19) modifications had high performance and can be used together with a non-transferred strand that is biotinylated to allow for immobilization on a fBLT (as outlined in FIG. 11).

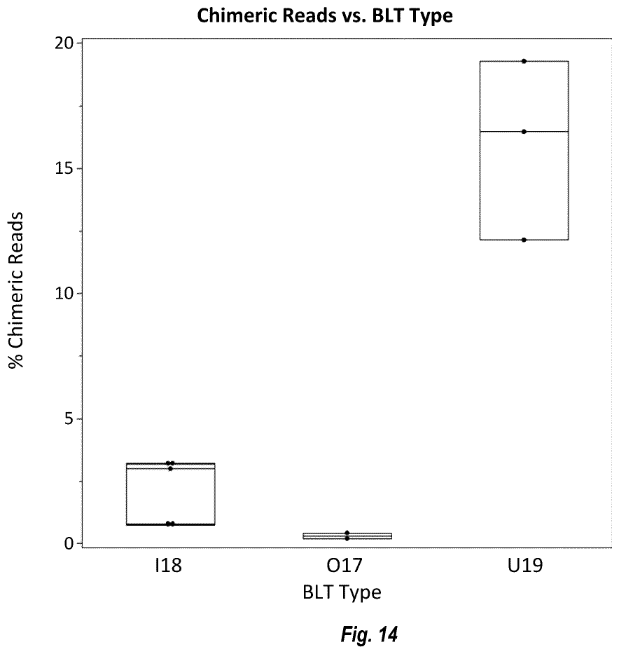

Although G19I showed an excellent profile, G19U (U19) led to a relatively high number of chimeric reads, wherein parts of the read map to different chromosomes, in relation to A18I (I18) and C17O (O17) modifications (FIG. 14). These chimeric reads may be due to a number of potential factors, such as the performance of different endonucleases (for example, cleavage of uracil by USER reagents may be less robust than cleavage of other modified nucleotides by their respective endonucleases). Chimeric reads are an undesired sequencing artifact, and thus a user may prefer to avoid uracil modifications (and subsequent cleavage of the mosaic end sequence with USER reagents) for certain methods to decrease the risk of chimeric reads.

Together, these data suggest that A18 and G19 modifications, such as G19I and A18I, may show high activity with fBLTs.

### Example 7. Comparison of fBLTs to Other Fragmentation Methods

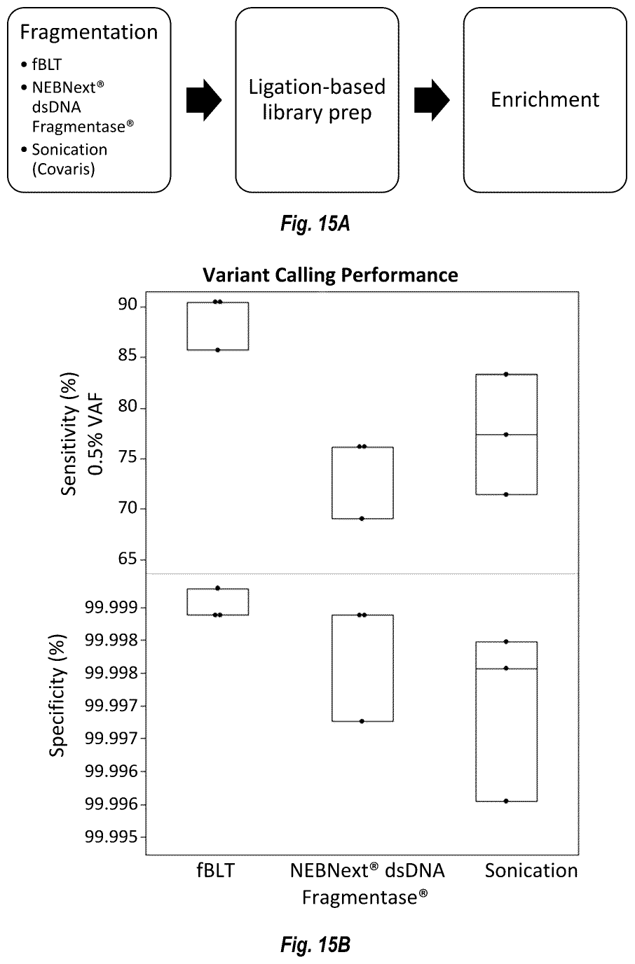

Fragmentation with A18I (I18) fBLTs was compared to fragmentation via NEBNext® dsDNA Fragmentase® or sonication using the workflow shown in FIG. 15A.

Sonication samples were sheared by a Covaris ultrasonicator (LE220 model) according to the manufacturer's recommended protocol designed for 175 bp fragments (see, for example, Quick Guide to DNA Shearing with LE220, Covaris, May 2020).

Sample fragmentation with NEBNExt dsDNA Fragmentase was performed according to the manufacturer's protocol and reagents. A time course study was performed using a variety of samples of interest to predetermine optimal sample incubation conditions. A 10-minute incubation at room temperature (approximately 20° C.) was the best single condition that could be utilized for all samples of interest.

Fragmentation with fBLTs was performed as described in Example 6.

End-repair and A-tailing was performed in the same manner for all fragments using Illumina reagents. The same pool of UMI-containing forked adapters were ligated with fragments prepared by each method. This outlines that an advantage of the present fBLT method is that it can use preexisting adapters that have been developed for other types of ligation-based library preparations.

Following PCR amplification, resulting libraries were enriched following the enrichment protocols set forth in the RNA Prep with Enrichment Reference Guide (Illumina Document No: 1000000124435), using the TruSight Cancer panel.

The sample used for assessment was a 50 ng input genomic DNA (gDNA) 1% mixture of NA12877 in NA12878 background (50 ng input). Based on this mixture, there are 84 expected heterozygous variants (leading to a 0.5% variant allele frequency (VAF)). Results are shown in FIG. 15B, with the fBLT method showing higher sensitivity and specificity as compared to either NEBNext® dsDNA Fragmentase® or sonication protocols.

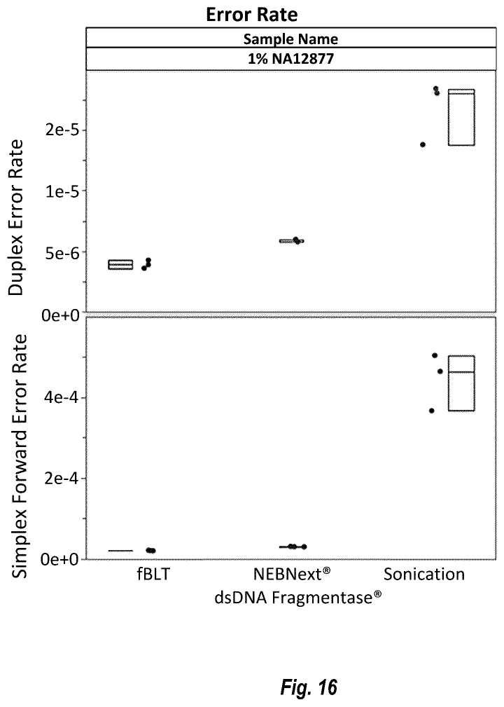

Error rates were also assessed with the different fragmentation methods. As shown in FIG. 16, substantially higher duplex error rate and simplex forward error rate were seen for sonicated samples, as compared to samples prepared with fBLTs or NEBNext® dsDNA Fragmentase®. Such increases in error rates generally indicate that there is greater noise in the data, i.e. more variability in the library fragments that were sequenced.

The stranded G>T error rate for samples prepared using fBLTs was 1.4×10⁻⁵, while this error rate was 70×10⁻⁵ for sample prepared via sonication. These data indicate that there was an approximately 50-fold reduction in false-positive G>T transversions for the fBLT method in comparison to the sonication method. The improved error rate with fBLTs is likely because the fBLT method avoids oxidative damage to guanine that may be induced by sonication.

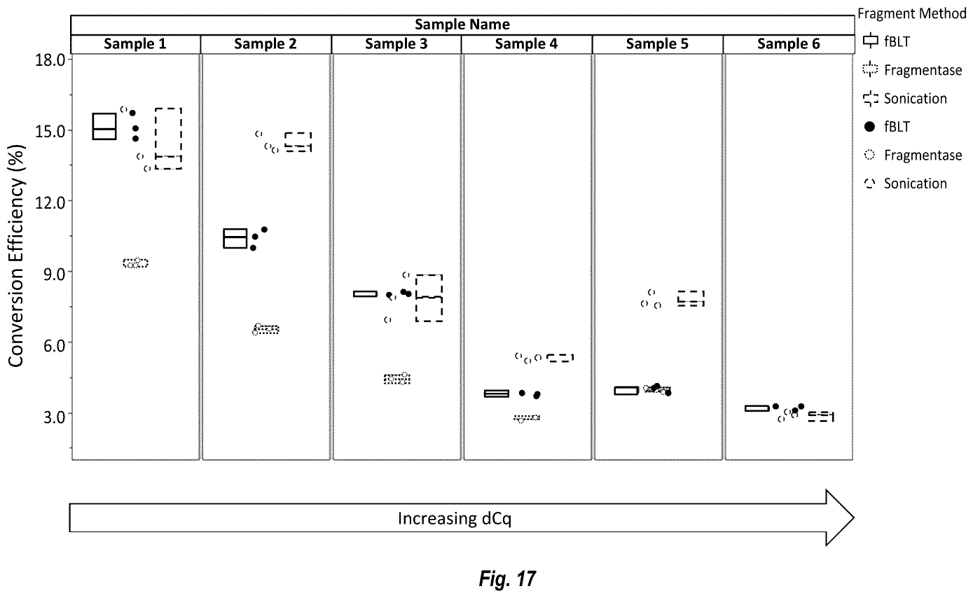

FIG. 17 shows that across a range of samples, the fBLT method outperformed the enzymatic NEBNext® dsDNA Fragmentase® method and gave similar library conversion efficiency as the sonication protocol.

Thus, fBLTs are a means of preparing libraries with advantages of improved sensitivity/specificity and reduced error rates as compared to other methods of library preparation with fragmentation that are currently used. Sample 1 in FIG. 17 represents a genomic DNA sample, while samples 2-6 represent formalin-fixed paraffin embedded (FFPE) samples. The higher conversion efficiency of Sample 1 compared to the other samples is a function of the higher quality of DNA in a genomic DNA sample as compared to FFPE samples, as is well-known in the field. The increase in dCq from Samples 2-6 shows that sample quality was worse for higher-numbered samples and that quality was higher for Sample 1 with genomic DNA.

FIG. 19 summarizes some advantages of the fBLT method, including that the user can choose a variety of different adapters to ligate onto fragments prepared with fBLTs based on a preferred downstream workflow. For example, a user wanting a streamlined workflow could use indexed forked adapters, which can avoid downstream PCR to incorporate index sequences. Similarly, a user wanting high sensitivity for calling different fragments could use adapters comprising UMIs (UMI adapters) such that amplicons of the same fragment can be identified from sequencing results after PCR amplification. Thus, fBLTs can combine the advantages of tagmentation for library preparation with the flexibility of ligation-based protocols, wherein a wide variety of different adapters can be incorporated into library fragments.

### Example 8. fBLTs for Use With Formalin-Fixed Paraffin Embedded Samples

A protocol was developed for preparing library fragments from formalin-fixed paraffin embedded (FFPE) samples using fBLTs. FFPE samples can contain critical information, such as the profile from a tumor sample, but FFPE material is often highly fragmented, which can interfere with standard library preparation protocols.

As shown in FIG. 18A, DNA is often partially fragmented within FFPE tissue. Standard tagmentation protocols require 2 tagmentation events per library fragment (i.e., a tagmentation event at each end of fragments). However, 2 tagmentation events with DNA from FFPE tissue can lead to a high ratio of very small fragments that are undesired for sequencing, due to this partial fragmentation of the starting DNA in the FFPE sample.

In contrast, fBLTs can be used to prepare singly tagmented fragments, i.e., fragments where the fBLT has only tagmented one end of the fragment, that can be rescued by ligation of adapters. After mosaic end cleavage, both ends of the fragments can be repaired and adapter ligated. In this way, library fragments from FFPE tissue can be generated with a single tagmentation event following by ligation at both ends of the fragments, leading to rescue of these fragments.

As shown in FIG. 18B, fragments prepared from DNA within FFPE can be rescued when prepared by single tagmentation events by fBLTs. Thus, a fBLT workflow can improve library preparation from FFPE tissue and other samples that may comprise partially fragmented DNA.

### Example 9. Workflows for fBLT Library Preparation and Optional Enrichment

Based on optimization experiments, a preliminary workflow for fBLT library preparation followed by enrichment was developed. This workflow is shown in FIG. 20. In summary, after tagmentation with a BLT, the tagmentation product is cleaned up and then the mosaic end (ME) is cleaved. After end repair and A-tailing, an adapter is ligated (wherein the user can choose the adapter, such as one comprising a UMI). If desired, a user can then perform solid-phase reversible immobilization (SPRI) bead purification, followed by indexing PCR and another SPRI bead purification. Such a workflow may take approximately 5.5 hours. The time for this workflow is similar to other ligation-based library preparation protocols.

If a user wishes to enrich the library, this can be performed such as hybridization followed by capture. Such a method may take approximately 5 hours.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A modified transposon end sequence comprising a mosaic end sequence, wherein the mosaic end sequence comprises one or more mutations as compared to a wild-type mosaic end sequence, wherein the mutation comprises a substitution with
a. a uracil;
b an inosine;
c. a ribose;
d. an 8-oxoguanine;
e. a thymine glycol;
f. a modified purine; or
g. a modified pyrimidine.

2. A transposome complex comprising:
a. a transposase;
b. a first transposon comprising a modified transposon end sequence comprising a uracil, an inosine, a ribose, an 8-oxoguanine, a thymine glycol, a modified purine, and/or a modified pyrimidine; and
c. a second transposon comprising a second transposon end sequence complementary to at least a portion of the first transposon end sequence.

3. A method of preparing double-stranded nucleic acid fragments comprising adapters comprising:
a. combining a sample comprising nucleic acid with transposome complexes comprising:
i. a transposase;
ii a first transposon comprising a modified transposon end sequence comprising a uracil, an inosine, a ribose, an 8-oxoguanine, a thymine glycol, a modified purine, and/or a modified pyrimidine; and
iii. a second transposon comprising a second transposon end sequence complementary to at least a portion of the first transposon end sequence;
b. preparing nucleic acid fragments;
c. combining the sample with (1) an endonuclease or (2) a combination of a DNA glycosylase and heat, basic conditions, or an endonuclease/lyase that recognizes abasic sites and cleaving the first transposon end at the uracil, inosine, ribose, 8-oxoguanine, thymine glycol, modified purine, and/or modified pyrimidine within the mosaic end sequence to remove all or part of the first transposon end from the nucleic acid fragments; and
d. ligating an adapter onto the 5′ and/or 3′ ends of the nucleic acid fragments.

4. The method of claim 3, wherein the modified purine is 3-methyladenine or 7-methylguanine and/or the modified pyrimidine is 5-methylcytosine, 5-formylcytosine, or 5-carboxycytosine.

5. The method of claim 3, wherein the nucleic acid is double-stranded DNA.

6. The method of claim 3, wherein the nucleic acid is RNA, and double-stranded cDNA or DNA:RNA duplexes are generated before combining with the transposome complexes.

7. The method of claim 3, wherein the all or part of the first transposon end that is cleaved is partitioned away from the rest of the sample.

8. The method of claim 3, further comprising filling in the 3′ ends of the fragments and phosphorylating the 3′ ends of fragments with a kinase before ligating, optionally wherein the filling in is performed with T4 DNA polymerase.

9. The method of claim 8, further comprising adding a single A overhang to the 3′ end of the fragments.

10. The method of claim 9, wherein a polymerase adds the single A overhang.

11. The method of claim 10, wherein the polymerase is (i) Taq or (ii) Klenow fragment, exo-.

12. The method of claim 3, wherein the fragments comprise 0-3 bases of the mosaic end sequence.

13. The method of claim 3, wherein preparing fragments leads to preparation of at least 50%, at least 60%, at least 70%, at least 80%, or at least 90% the number of fragments, as compared with preparing fragments with a transposome complex that comprises a first transposon comprising a transposon end sequence comprising a wildtype mosaic end sequence comprising SEQ ID No: 1.

14. The method of claim 3, further comprising sequencing the fragments after ligating the adapter, optionally wherein:
a. the method does not require amplification of fragments before sequencing or fragments are amplified before sequencing; or
b. fragments are amplified before sequencing.

15. The method of claim 14, further comprising enriching fragments of interest after ligating the adapter and before sequencing.

16. The method of claim 3, wherein:
a. the modified transposon end sequence comprises a uracil and the combination of a DNA glycosylase and an endonuclease/lyase that recognizes abasic sites is a uracil-specific excision reagent (USER), optionally wherein the USER is a mixture of uracil DNA glycosylase and endonuclease VIII or endonuclease III;
b. the modified transposon end sequence comprises an inosine and the endonuclease is endonuclease V;
c. the modified transposon end sequence comprises a ribose and the endonuclease is RNAse HII;
d. the modified transposon end sequence comprises a 8-oxoguanine and the endonuclease is formamidopyrimidine-DNA glycosylase (FPG) or oxoguanine glycosylase (OGG);
e. the modified transposon end sequence comprises a thymine glycol and the DNA glycosylase is endonuclease EndoIII (Nth) or Endo VIII;
f. the modified transposon end sequence comprises a modified purine and the DNA glycosylase is human 3-alkyladenine DNA glycosylase and the endonuclease is endonuclease III or VIII, optionally wherein the modified purine is 3-methyladenine or 7-methylguanine; or
g. the modified transposon end sequence comprises a modified pyrimidine, optionally wherein the modified pyrimidine is 5-methylcytosine, 5-formylcytosine, or 5-carboxycytosine, and:
i. the DNA glycosylase is thymine-DNA glycosylase (TDG) or mammalian DNA glycosylase-methyl-CpG binding domain protein 4 (MBD4) and the endonuclease/lyase that recognizes abasic sites is the endonuclease is endonuclease III or VIII; or
ii. the endonuclease is DNA glycosylase/lyase ROS1 (ROS1).

17. The method of claim 3, wherein the first transposon comprises a modified transposon end sequence comprising more than one mutation chosen from a uracil, an inosine, a ribose, 8-oxoguanine, a thymine glycol, a modified purine, or a modified pyrimidine and the (1) an endonuclease or (2) a combination of a DNA glycosylase and heat, basic conditions, or an endonuclease/lyase that recognizes abasic sites is an enzyme mixture, optionally wherein the modified purine is 3-methyladenine or 7-methylguanine and/or the modified pyrimidine is 5-methylcytosine, 5-formylcytosine, or 5-carboxycytosine.

18. The method of claim 3, wherein cleaving the first transposon end generates a sticky end for ligating the adapter, optionally wherein the sticky end is longer than one base.

19. The method of claim 3, wherein:
a. the adapter comprises a double-stranded adapter;
b. adapters are added to the 5′ and 3′ end of fragments, optionally wherein the adapters added to the 5′ and 3′ end of the fragments are different;
c. the adapter comprises a unique molecular identifier (UMI), primer sequence, anchor sequence, universal sequence, spacer region, index sequence, capture sequence, barcode sequence, cleavage sequence, sequencing-related sequence, and combinations thereof;
d. the adapter comprises a UMI, optionally wherein an adapter comprising a UMI is ligated to both the 3′ and 5′ end of fragments; and/or
e. the adapter is a forked adapter.

20. The method of claim 3, wherein:
a. the ligating is performed with a DNA ligase;
b the method is performed in a single reaction vessel;
c. the density of transposomes immobilized on the solid surface is selected to modulate fragment size and library yield of the immobilized fragments;
d. the method allows for bead-based normalization;
e. the sample comprises partially fragmented DNA;
f. the sample is formalin fixed paraffin embedded tissue or cell-free DNA; and/or
g. the library comprises fragments prepared by a single tagmentation event.


SEQUENCE LISTING (22 sequences)

SEQ ID NO: 1 - DNA, 19 bp, synthetic construct
  source 1..19: mol_type = other DNA
agatgtgtataagagacag

SEQ ID NO: 2 - DNA, 19 bp, synthetic construct
  source 1..19: mol_type = other DNA
ctgactcttatacacaagt

SEQ ID NO: 3 - DNA, 19 bp, synthetic construct
  source 1..19: mol_type = other DNA
ctgtctcttgatcagatct

SEQ ID NO: 4 - DNA, 19 bp, synthetic construct
  source 1..19: mol_type = other DNA
ctgtctcttatacacatct

SEQ ID NO: 5 - DNA, 19 bp, synthetic construct
  source 1..19: mol_type = other DNA
  modified_base 16: mod_base = OTHER; note = uracil
agatgtgtataagagtcag

SEQ ID NO: 6 - DNA, 19 bp, synthetic construct
  source 1..19: mol_type = other DNA
ctgactcttatacacatct

SEQ ID NO: 7 - DNA, 19 bp, synthetic construct
  source 1..19: mol_type = other DNA
  modified_base 17: mod_base = OTHER; note = uracil
agatgtgtataagagatag

SEQ ID NO: 8 - DNA, 19 bp, synthetic construct
  source 1..19: mol_type = other DNA
ctatctcttatacacatct

SEQ ID NO: 9 - DNA, 19 bp, synthetic construct
  source 1..19: mol_type = other DNA
  modified_base 18: mod_base = OTHER; note = uracil
agatgtgtataagagactg

SEQ ID NO: 10 - DNA, 19 bp, synthetic construct
  source 1..19: mol_type = other DNA
cagtctcttatacacatct

SEQ ID NO: 11 - DNA, 14 bp, synthetic construct
  source 1..14: mol_type = other DNA
tcgtcggcagcgtc

SEQ ID NO: 12 - DNA, 15 bp, synthetic construct
  source 1..15: mol_type = other DNA
gtctcgtgggctcgg

SEQ ID NO: 13 - DNA, 19 bp, synthetic construct
  modified_base 1: mod_base = OTHER; note = 5' phosphate
  modified_base 19: mod_base = OTHER; note = 3' biotin
  source 1..19: mol_type = other DNA
ctgtctcttatacacatct

SEQ ID NO: 14 - DNA, 19 bp, synthetic construct
  modified_base 19: mod_base = i
  source 1..19: mol_type = other DNA
agatgtgtataagagacan

SEQ ID NO: 15 - DNA, 19 bp, synthetic construct
  source 1..19: mol_type = other DNA
  modified_base 19: mod_base = OTHER; note = uracil
agatgtgtataagagacat

SEQ ID NO: 16 - DNA, 19 bp, synthetic construct
  modified_base 16: mod_base = OTHER; note = 8-oxo-guanine
  source 1..19: mol_type = other DNA
agatgtgtataagaggcag

SEQ ID NO: 17 - DNA, 19 bp, synthetic construct
  modified_base 17: mod_base = OTHER; note = 8-oxo-guanine
  source 1..19: mol_type = other DNA
agatgtgtataagagagag

SEQ ID NO: 18 - DNA, 19 bp, synthetic construct
  modified_base 18: mod_base = OTHER; note = 8-oxo-guanine
  source 1..19: mol_type = other DNA
agatgtgtataagagacgg

SEQ ID NO: 19 - DNA, 19 bp, synthetic construct
  modified_base 19: mod_base = OTHER; note = 8-oxo-guanine
  source 1..19: mol_type = other DNA
agatgtgtataagagacag

SEQ ID NO: 20 - DNA, 19 bp, synthetic construct
  modified_base 16: mod_base = i
  source 1..19: mol_type = other DNA
agatgtgtataagagncag

SEQ ID NO: 21 - DNA, 19 bp, synthetic construct
  modified_base 17: mod_base = i
  source 1..19: mol_type = other DNA
agatgtgtataagaganag

SEQ ID NO: 22 - DNA, 19 bp, synthetic construct
  modified_base 18: mod_base = i
  source 1..19: mol_type = other DNA
agatgtgtataagagacng